(19) United States
(12) Patent Application Publication
(10) Pub. No.: US 2022/0096411 A1
(43) Pub. Date: Mar. 31, 2022

(54) COMBINING METABOLIC STIMULATION AND AMINO ACIDS TO SENSITIZE TOLERANT BACTERIA TO ANTIBIOTICS

(71) Applicants: THE BROAD INSTITUTE, INC., Cambridge, MA (US); MASSACHUSETTS INSTITUTE OF TECHNOLOGY, Cambridge, MA (US)

(72) Inventors: Sarah Bening, Cambridge, MA (US); Meagan Hamblin, Cambridge, MA (US); Ian Andrews, Cambridge, MA (US); Allison Lopatkin, Cambridge, MA (US); James Collins, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 17/425,438

(22) PCT Filed: Jan. 23, 2020
(86) PCT No.: PCT/US2020/014786

§ 371 Date: Jul. 23, 2021

Related U.S. Application Data
(60) Provisional application No. 62/796,360, filed on Jan. 24, 2019.

(51) Int. Cl. A61K 31/198; A61K 31/431; A61K 31/424; A61K 31/46; A61K 31/546; A61K 31/407

(52) U.S. Cl. CPC A61K 31/198; A61K 31/431; A61K 31/407; A61K 31/46; A61K 31/546; A61K 31/424

(57) ABSTRACT

The present disclosure provides compositions and methods capable of potentiating the effects of antibiotics against bacterial infections that either have developed, or that possess the potential to develop, antibiotic tolerance. Methods of sensitizing antibiotic tolerant bacteria to antibiotics, as well as pharmaceutical compositions and therapeutic/prophylactic methods directed at antibiotic tolerant bacteria are also provided.

### CROSS REFERENCE TO RELATED APPLICATION

This application claims the benefit of U.S. Provisional Application No. 62/796,360, filed Jan. 24, 2019, entitled “Combining Metabolic Stimulation and Amino Acids to Sensitize Tolerant Bacteria to Antibiotics,” the entire contents of which are incorporated herein by reference.

### STATEMENT REGARDING FEDERALLY SPONSORED RESEARCH

This invention was made with government support under Grant No. 1122374 awarded by the National Science Foundation and under Grant No. HDTRA1-15-1-0051 awarded by the Department of Defense. The government has certain rights in the invention.

### FIELD OF THE INVENTION

The invention relates generally to methods and compositions for treatment and prevention of antibiotic tolerant bacteria.

### BACKGROUND OF THE INVENTION

The clinically observed phenomenon of bacteria developing tolerance to the lethal effects of antibiotics produces chronic and costly infections that possess an increased potential to select for antibiotic resistance. Antibiotic tolerance emanating from administration of high antibiotic concentrations can therefore lead to chronic infections and resistance. A need exists for compositions and/or improved approaches that can sensitize antibiotic-tolerant bacteria to antibiotics, both in vitro and in vivo.

### BRIEF SUMMARY OF THE INVENTION

The current disclosure relates, at least in part, to compositions and methods for sensitizing bacteria—particularly bacteria that have developed antibiotic tolerance (due to being in stationary phase or for other reason)—to antibiotic contact and/or treatment. In particular, an approach of metabolic stimulation and contact of antibiotic tolerant bacteria with D-amino acids was identified as potentiating the antimicrobial activity of certain antibiotics, an effect that could also be more broadly applied via optional inclusion of a β-lactamase inhibitor, where relevant to anti-bacterial compositions and approaches of the instant disclosure.

In one aspect, the instant disclosure provides a pharmaceutical composition that includes (a) a metabolic stimulant; (b) a D-amino acid; (c) an antibiotic or a pharmaceutically acceptable salt thereof, and a pharmaceutically acceptable carrier.

In one embodiment, the metabolic stimulant is a carbon source. Optionally the metabolic stimulant is citrate, propionic acid, succinate, pyruvate, fumarate, acetate, mannitol, glycerol, arabinose, fructose, xylose, glucose, ribose, gluconate, a L-amino acid (examples including L-alanine and L-serine, etc.) or a D-amino acid (specific examples including D-alanine, D-serine, etc.). Optionally, other art-recognized carbon sources, including those specifically recited elsewhere herein, are employed as a metabolic stimulant.

In another embodiment, the D-amino acid is D-alanine, D-cysteine, D-aspartic acid, D-glutamic acid, D-phenylalanine, D-histidine, D-isoleucine, D-lysine, D-leucine, D-methionine, D-asparagine, D-norleucine, D-glutamine, D-arginine, D-serine, D-threonine, D-valine, D-tryptophan, D-asparagine, D-phenylglycine, D-tyrosine, D-alpha-aminobutyric acid, or D-alpha-aminopimelic acid, or a combination thereof. (While initial results for D-proline were described herein as less robust than for other D-amino acids, use of D-proline is nonetheless contemplated in certain embodiments.)

In certain embodiments, the antibiotic is a β-lactam antibiotic, an aminoglycoside antibiotic and/or a quinolone antibiotic. Optionally, the β-lactam antibiotic is a penicillin derivative (e.g., Benzathine penicillin (benzathine & benzylpenicillin), Benzylpenicillin (penicillin G), Phenoxymethylpenicillin (penicillin V), Procaine penicillin (procaine & benzylpenicillin), Pheneticillin, Cloxacillin, Dicloxacillin, Flucloxacillin, Methicillin, Nafcillin, Oxacillin, Temocillin, Amoxicillin, Ampicillin, Mecillinam, Carbenicillin, Ticarcillin, Azlocillin, Mezlocillin, and/or Piperacillin); a cephalosporin (e.g., Cefazolin, Cephalexin, Cephalosporin C, Cephalothin, Cefaclor, Cefamandole, Cefuroxime, Cefotetan, Cefoxitin, Cefixime, Cefotaxime, Cefpodoxime, Ceftazidime, Ceftriaxone, Cefepime, Cefpirome, Cefsulodin and/or Ceftaroline); a monobactam (e.g., Aztreonam, Tigemonam, Nocardicin A, and Tabtoxinine β-lactam), and/or a carbapenem or penem (e.g., Biapenem, Doripenem, Ertapenem, Faropenem, Imipenem, Meropenem, Panipenem, Razupenem, Tebipenem, and/or Thienamycin). In some embodiments, combination β-lactam antibiotics can be employed, including, for example and without limitation, Amoxicillin/clavulanic acid, Imipenem/cilastatin, Ampicillin/flucloxacillin, Ampicillin/sulbactam (Sultamicillin), Ceftazidime/avibactam, Piperacillin/tazobactam, Ceftolozane/tazobactam, cefoperazone/sulbactam, and/or Meropenem/vaborbactam. It is also contemplated that the compositions and methods of the instant disclosure could be effectively used with other cell wall-active antibiotics, including, for example and without limitation, NAM synthesis inhibitors (e.g., Fosfomycin), DADAL/AR inhibitors (e.g., Cycloserine), bactoprenol inhibitors (e.g., Bacitracin), PG chain elongation inhibitors (e.g., Vancomycin (Oritavancin, Telavancin), Teicoplanin (Dalbavancin), Ramoplanin), polymyxins/detergent (e.g., Colistin, Polymyxin B), depolarizing agents (e.g., Daptomycin), NAM-NAG hydrolysis agents (e.g., lysozyme), Tyrothricin (e.g., Gramicidin, Tyrocidine), Isoniazid, and/or Teixobactin. (For cell-wall active antibiotics, see also Silver et al. Current Opinion in Microbiology, 6: 431-438, which is incorporated by reference herein in its entirety.)

In one embodiment, the pharmaceutical composition further includes a β-lactamase inhibitor. Optionally, the β-lactamase inhibitor is sulbactam, tebipenem, a Boron based transition state inhibitor (e.g., Ec19), clavulanic acid, tazobactam, avibactam and/or relebactam.

In certain embodiments, the antibiotic is present in an amount between 0.1 g and 2.0 g.

In some embodiments, the D-amino acid is provided in an amount sufficient to potentiate the antibiotic to kill at least 80% of a target population of bacteria that possess antibiotic tolerance.

Another aspect of the instant disclosure provides a pharmaceutical composition that includes: (a) a β-lactamase inhibitor; (b) a metabolic stimulant and/or a D-amino acid; and a pharmaceutically acceptable carrier.

An additional aspect of the disclosure provides a method for sensitizing a bacteria to an antibiotic, the method involving contacting the bacteria with a metabolic stimulant and a D-amino acid, thereby sensitizing the bacteria to the antibiotic.

In one embodiment, the bacteria exhibits tolerance or resistance to the antibiotic.

In certain embodiments, the bacteria is an Escherichia coli, Klebsiella and/or a Mycobacteria. Optionally, the Klebsiella is a Klebsiella pneumoniae and/or the Mycobacteria is a Mycobacterium smegmatis or a Mycobacterium tuberculosis.

In some embodiments, the method further involves contacting the bacteria with a β-lactamase inhibitor.

Another aspect of the instant disclosure provides a method for sensitizing a bacteria that expresses β-lactamase to an antibiotic, the method involving contacting the bacteria with a β-lactamase inhibitor and a metabolic stimulant and/or a D-amino acid, thereby sensitizing the bacteria to the antibiotic.

An additional aspect of the instant disclosure provides a method for treating or preventing a bacterial infection in a subject, the method involving administering a pharmaceutical composition of the disclosure to a subject having or at risk of developing a bacterial infection, thereby treating or preventing the bacterial infection in the subject.

In one embodiment, the subject is human.

In certain embodiments, the bacterial infection is an antibiotic tolerant or antibiotic resistant bacterial infection.

Another aspect of the instant disclosure provides a method for treating or preventing a bacterial infection in a subject, where bacteria of the bacterial infection express β-lactamase and exhibit antibiotic tolerance, the method involving administering a pharmaceutical composition that includes (a) a β-lactamase inhibitor; (b) a metabolic stimulant and/or a D-amino acid; and (c) a pharmaceutically acceptable carrier to the subject in an amount sufficient to treat or prevent the bacterial infection in the subject.

An additional aspect of the instant disclosure provides a kit that includes a metabolic stimulant, a D-amino acid, a β-lactam antibiotic, and instructions for its use.

In one embodiment, the kit further includes a β-lactamase inhibitor.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “infection” as used herein includes presence of bacteria, in or on a subject, which, if its growth were inhibited or if killing and/or clearing of the bacteria from a site of infection were to occur, would result in a benefit to the subject. The term “infection” therefore refers to any undesirable form of bacteria that is present on or in a subject. As such, the term “infection” in addition to referring to the presence of bacteria also refers to normal flora, which are not desirable. The term “infection” includes infection caused by bacteria.

The term “treat”, “treating” or “treatment” as used herein refers to administering a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection. The term “therapeutic treatment” refers to administering treatment to a subject already suffering from infection. The term “treat”, “treating” or “treatment” as used herein also refers to administering compositions or one or more of pharmaceutically active ingredients discussed herein, with or without additional pharmaceutically active or inert ingredients, in order to: (i) reduce or eliminate either a bacterial infection or one or more symptoms of the bacterial infection, or (ii) retard the progression of a bacterial infection or of one or more symptoms of the bacterial infection, or (iii) reduce the severity of a bacterial infection or of one or more symptoms of the bacterial infection, or (iv) suppress the clinical manifestation of a bacterial infection, or (v) suppress the manifestation of adverse symptoms of the bacterial infection.

The term “pharmaceutically effective amount” or “therapeutically effective amount” or “effective amount” as used herein refers to an amount, which has a therapeutic effect or is the amount required to produce a therapeutic effect in a subject. For example, a therapeutically or pharmaceutically effective amount of an antibiotic or a pharmaceutical composition is the amount of the antibiotic or the pharmaceutical composition required to produce a desired therapeutic effect as may be judged by clinical trial results, model animal infection studies, and/or in vitro studies (e.g., in agar or broth media). The pharmaceutically effective amount depends on several factors, including but not limited to, the microorganism (e.g., bacteria) involved, characteristics of the subject (for example height, weight, sex, age and medical history), severity of infection and the particular type of the antibiotic used. For prophylactic treatments, a therapeutically or prophylactically effective amount is that amount which would be effective to prevent a microbial (e.g. bacterial) infection.

The term “administration” or “administering” includes delivery of a composition or one or more pharmaceutically active ingredients to a subject, including for example, by any appropriate methods, which serves to deliver the composition or its active ingredients or other pharmaceutically active ingredients to the site of the infection. The method of administration may vary depending on various factors, such as for example, the components of the pharmaceutical composition or the type/nature of the pharmaceutically active or inert ingredients, the site of the potential or actual infection, the microorganism involved, severity of the infection, age and physical condition of the subject and a like. Some non-limiting examples of ways to administer a composition or a pharmaceutically active ingredient to a subject according to this invention includes oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash. In case of a pharmaceutical composition that comprises more than one ingredient (active or inert), one of way of administering such composition is by admixing the ingredients (e.g. in the form of a suitable unit dosage form such as tablet, capsule, solution, powder and a like) and then administering the dosage form. Alternatively, the ingredients may also be administered separately (simultaneously or one after the other) as long as these ingredients reach beneficial therapeutic levels such that the composition as a whole provides a synergistic and/or desired effect.

The term “growth” as used herein refers to a growth of one or more microorganisms and includes reproduction or population expansion of the microorganism (e.g., bacteria). The term also includes maintenance of on-going metabolic processes of a microorganism, including processes that keep the microorganism alive.

The term, “effectiveness” as used herein refers to ability of a treatment or a composition or one or more pharmaceutically active ingredients to produce a desired biological effect in a subject. For example, the term “antibiotic effectiveness” of a composition or a beta-lactam antibiotic refers to the ability of the composition or the beta-lactam antibiotic to prevent or treat the microbial (e.g., bacterial) infection in a subject.

The term “synergistic” or “synergy” as used herein refers to the interaction of two or more agents so that their combined effect is greater than their individual effects.

The term “antibiotic” as used herein refers to any substance, compound or a combination of substances or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibiotic” also refers to compounds capable of decreasing infectivity or virulence of bacteria.

The term “β-lactam antibiotic” as used herein refers to compounds with antibiotic properties and containing a β-lactam ring in their molecular structure.

The term “beta-lactamase” as used herein refers to any enzyme or protein or any other substance that breaks down a beta-lactam ring. The term “beta-lactamase” includes enzymes that are produced by bacteria and have the ability to hydrolyze the beta-lactam ring in a beta-lactam antibiotic, either partially or completely.

The term “beta-lactamase inhibitor” as used herein refers to a compound capable of inhibiting activity of one or more beta-lactamase enzymes, either partially or completely.

The term “D-amino acid” as used herein refers to the dextrorotatory (clockwise rotating) enantiomeric form of an amino acid. D-amino acids include D-forms of naturally occurring amino acids, synthetic amino acids, and modified and/or derivatized forms of natural and synthetic amino acids that maintain a dextrorotary enantiomeric form.

As used herein, the term “metabolic stimulant” refers to a sugar, metabolite or other carbon source used in metabolism by a microbe building its biomass. “Carbon source” as used herein refers to a carbon-containing compound that is used by an organism as the source of carbon for building its biomass. In certain embodiments, an exemplary metabolic stimulant is a sugar.

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tissue” is intended to mean an aggregation of cells, and, optionally, intercellular matter. Typically the cells in a tissue are not free floating in solution and instead are attached to each other to form a multicellular structure. Exemplary tissue types include muscle, nerve, epidermal and connective tissues.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A and 1B show that metabolite supplementation restored β-lactam antibiotic sensitivity to tolerant bacteria. FIG. 1A shows the effect of different tested concentrations of ampicillin (a β-lactam antibiotic) on the survival of the bacteria at 100% (left), 10% (center), and 1% (right) LB medium. FIG. 1B shows metabolite-enabled minimal inhibitory concentrations (MICs) with β-lactams from four different generations of penicillins—ampicillin, carbenicillin, mecillinam, and penicillin. The indicated carbon sources are succinate (SUC), pyruvate (PYR), acetate (ACE), mannitol (MAN), glycerol (GLY), L-alanine (L-ALA), arabinose (ARA), fructose (FRC), xylose (XYL), glucose (GLC), ribose (RIB), gluconate (GLN), L-serine (L-SER), D-serine (D-SER), and D-alanine (D-ALA).

FIGS. 2A to 2G show that D-amino acids potentiated β-lactam antibiotic efficacy against tolerant bacteria without stimulating growth. FIG. 2A shows that in a genetic knockout strain (ΔdadA) that is unable to catabolize D-alanine into pyruvate, tolerant bacteria were equally insensitive to ampicillin, regardless of whether supplementation with a D-amino acid (D-alanine) or an L-amino acid (L-alanine) occurred. In contrast, when the ΔdadA strain was supplemented with pyruvate (thereby effectively removing the catabolic impact of the dadA deletion), D-alanine supplementation was observed to potentiate sensitivity to ampicillin, whereas no such potentiation was observed for L-alanine. FIG. 2B shows the changes in observed ampicillin minimum inhibitory concentration (MIC) values observed in the presence of various indicated carbon sources (propionate (PRP), succinate (SUC), xylose (XYL), arabinose (ARA), gluconate (GLN), ribose (RIB), glycerol (GLY), mannitol (MAN), fructose (FRC), pyruvate (PRV), and glucose (GLC)), when supplemented with either D-alanine (red) or L-alanine (blue). FIG. 2C shows the changes in ampicillin MIC values observed in the presence of pyruvate as the metabolic stimulant (carbon source) and either L-amino acid (blue) or D-amino acid (red) as indicated—specifically, MIC values were obtained for D- or L-alanine (in the presence of a dadA mutant), D- or L-proline, D- or L-leucine, D- or L-isoleucine, D- or L-threonine, D- or L-norleucine, D- or L-valine, and D- or L-methionine. FIG. 2D shows the changes in ampicillin MIC values observed in the presence of indicated D-amino acids (D-alanine (in the presence of a dadA mutant), D-proline, D-leucine, D-isoleucine, D-threonine, D-norleucine, D-valine, and D-methionine, respectively), either in the presence (red) or absence (black) of pyruvate as a metabolic stimulant (carbon source). FIG. 2E and FIG. 2F show that observed potentiation by D-amino acids was specific to β-lactam antibiotics (e.g., ampicillin), at least as compared to D-cycloserine (which acts to disrupt early cytosolic peptidoglycan synthesis), ciprofloxacin (a quinolone inhibitor of DNA gyrase), and gentamicin (an aminoglycoside inhibitor of ribosomes). FIG. 2E shows results obtained with pyruvate used as a metabolic stimulant, while FIG. 2F shows results obtained without a metabolic stimulant. FIG. 2G shows that metabolic stimulation by pyruvate and potentiation by D-amino acids sensitized tolerant bacteria to the β-lactam antibiotics imipenem and piperacillin.

FIGS. 3A to 3I show that the metabolite supplementation and D-amino acid administration approaches of the instant disclosure were generalizable to other conditions and to β-lactamase producing pathogens. FIG. 3A shows that supplementation with pyruvate restored ampicillin lethality against stationary phase cultures of MG1655 in MOPS-rich medium, and D-methionine further potentiated the effects of ampicillin. FIG. 3B shows that pyruvate and D-methionine in combination similarly sensitized a different E. coli strain, BW25113, grown in LB medium. FIG. 3C shows that in tolerant cultures, the β-lactamase inhibitor sulbactam alone was unable to restore sensitivity to ampicillin, yet combining sulbactam with pyruvate successfully restored sensitivity to cultures of resistant E. coli in stationary phase, and D-methionine further potentiated killing by lower drug concentrations (left panel). Potentiation was not observed in the absence of sulbactam (right panel). FIGS. 3D and 3E show that the combination of all three components—pyruvate (FIG. 3D) or glucose (FIG. 3E), D-methionine, and sulbactam—sensitized stationary phase cultures of a β-lactamase-producing K. pneumoniae isolate to ampicillin. FIGS. 3F and 3G show that the combination of all three components—glucose (FIG. 3F) or glycerol (FIG. 3G), D-methionine, and sulbactam—also sensitized stationary phase cultures of a β-lactamase-producing M. smegmatis isolate to ampicillin. FIGS. 3H and 3I show that the combination of all three components—glucose (FIG. 3H) or glycerol (FIG. 3I), D-methionine, and clavulanic acid—sensitized stationary phase cultures of a β-lactamase-producing M. smegmatis isolate to amoxicillin. The three component approach of the instant disclosure exhibited a synergistic potentiation effect, as compared to individual or pairwise combinations (e.g., pyruvate and D-methionine alone) of components.

FIGS. 4A and 4B show potentiation of ampicillin across indicated metabolic stimulants and observed MICs for tested β-lactam antibiotics, which tended to be independent of bacterial population density (biomass accumulation, as measured by OD600). FIG. 4A shows a heat map that demonstrates that many metabolic stimulants potentiated ampicillin activity. FIG. 4B shows for the metabolites of FIG. 1B above the relationship between metabolite-enabled MIC and metabolite-stimulated biomass accumulation (in the absence of drug) for the β-lactam antibiotics ampicillin, carbenicillin, mecillinam, and penicillin.

FIG. 5 shows that individual carbon sources—from top left to bottom right, glucose, mannitol, gluconate, fructose, arabinose, glycerol, and xylose—exhibited varying abilities to restore β-lactam antibiotic sensitivity to tolerant cultures of MG1655 E. coli grown in 100% LB medium, as indicated by colony-forming units (CFU)/mL. Experiments with glucose, gluconate, fructose, arabinose, glycerol, and xylose were performed in replicates of 7, while experiments with mannitol were performed in replicates of 6, and experiments with glycerol were performed in replicates of 5. Data are presented as the mean (thick lines) with the range given by the shaded region and individual replicates shown as thin lines.

FIGS. 6A to 6D show that the combination of xylose and D-methionine restored sensitivity of tolerant MG1655 E. coli cultures grown in 100% LB medium, as indicated by CFU/mL, to low concentrations of four different β-lactam antibiotics. FIG. 6A shows that the combination of xylose and D-methionine restored the sensitivity of tolerant MG1655 E. coli cultures to ampicillin. Experiments were performed in duplicate. Data are presented as the mean (thick lines) with the range given by the shaded region and individual replicates shown as thin lines. FIG. 6B shows that the combination of xylose and D-methionine restored the sensitivity of tolerant MG1655 E. coli cultures to cefsulodin. Experiments were performed in triplicate. Data are again presented as the mean (thick lines) with the range given by the shaded region and individual replicates shown as thin lines. FIG. 6C shows that the combination of xylose and D-methionine restored the sensitivity of tolerant MG1655 E. coli cultures to mecillinam. Experiments were performed in triplicate. Data are presented as the mean (thick lines) with the range given by the shaded region and individual replicates shown as thin lines. FIG. 6D shows that the combination of xylose and D-methionine restored the sensitivity of tolerant MG1655 E. coli cultures to piperacillin. Experiments were performed in triplicate. Data are presented as the mean (thick lines) with the range given by the shaded region and individual replicates shown as thin lines.

FIGS. 7A and 7B show the effect of carbon sources and D-methionine on bacterial growth and population density as measured by colony-forming units (CFU). FIG. 7A shows that the carbon sources glucose, pyruvate, and xylose, did not lead to an increase in population density when added to cultures grown in 100% LB medium (left), but did stimulate growth and an increase in population density for cultures grown in 1% LB medium (right). FIG. 7B shows that D-methionine did not increase population density when used alone or in combination with xylose. Data are presented as the mean (thick lines) with the range given by the shaded region.

### DETAILED DESCRIPTION OF THE INVENTION

The present disclosure is directed, at least in part, to the discovery that contact of populations of antibiotic tolerant bacteria with a growth-promoting carbon source (e.g., glucose, pyruvate, etc.) and a D-amino acid (e.g., D-methionine, D-alanine, etc.) was remarkably effective at sensitizing such antibiotic-tolerant bacteria to an antibiotic (particularly a β-lactam antibiotic for β-lactam antibiotic tolerant bacteria), as compared to contacting such bacteria with such an antibiotic absent contact with the D-amino acid or absent contact with the growth-promoting carbon source. In particular, metabolic stimulation was identified as sensitizing tolerant bacteria to β-lactams, which constitute one of the oldest and most widely used classes of antimicrobials. D-amino acids were identified as further enhancing metabolite-enabled sensitization, and it was further identified that metabolic stimulation and/or D-amino acids could be combined with β-lactamase inhibitors, thereby promoting sensitization of resistant isolates of Escherichia coli, Klebsiella pneumoniae and Mycobacterium smegmatis to β-lactam antibiotics.

The instant disclosure therefore provides compositions capable of potentiating the effects of antibiotics against bacterial infections that have developed, or that are capable of developing, antibiotic tolerance, absent contact with such compositions of the disclosure. Methods of sensitization of antibiotic tolerant bacteria, as well as pharmaceutical compositions and therapeutic/prophylactic methods directed at antibiotic tolerant bacteria are also provided.

Bacteria can become tolerant to antibiotic treatment. Tolerance specifically refers to an inability of high concentrations of antibiotics—typically lethal concentrations that are above the growth-inhibitory threshold for a given strain—to kill bacteria. Tolerant bacterial infections are believed to contribute to recurrent infections which then take longer to treat, driving up treatment costs.

Effective treatment of bacterial infections is limited when bacteria are able to evade antibiotic action. Common mechanisms of antibiotic resistance are well understood in the art, and adjuvants and multidrug strategies targeting these resistance mechanisms are being developed and deployed clinically (1-4). To date, less has been known about mechanisms bacteria use to survive typically lethal antibiotic challenge—evasion mechanisms classified as antibiotic tolerance or persistence (5)—and treatment strategies targeting these survival mechanisms are only beginning to be developed (6). Addressing bacterial survival mechanisms is crucial for both current and future treatment efficacy, as recent in vitro evolution results have revealed that antibiotic tolerance provides a reservoir from which antibiotic resistance can emerge (7).

Metabolism has been described to play a central role in antibiotic lethality (8), and because nutrient limitation leads to multidrug tolerance (9-11), stationary phase has in many instances been used as a model to study tolerance reversion strategies. Indeed, stationary phase bacteria have been previously described as tolerant to most antibiotics: replenishing nutrients that are missing in stationary phase (oxygen and carbon) has been described to restore sensitivity to quinolone and aminoglycoside antibiotics (11). As described herein, stationary phase provides a model for study of β-lactam antibiotics, which constitute one of the oldest and most widely used drug classes.

β-lactam antibiotics inhibit the formation of 4-3 crosslinks by essential D, D-transpeptidases in bacterial peptidoglycan, and by blocking this reaction β-lactams induce an energy-demanding futile cycle of peptidoglycan intermediates that contributes to cell death (12). Stationary phase bacteria are tolerant to β-lactam antibiotics despite measurable peptidoglycan activity. Peptidoglycan structure is sensitive to β-lactams in stationary phase (13), and resting cells are also able to incorporate into peptidoglycan non-canonical D-amino acids (NCDAAs; 14, 15), which have previously been shown to be synergistic with β-lactams in other conditions (16). While metabolic stimulation has been able to sensitize tolerant, stationary phase bacteria to other classes of antibiotics, these approaches have thus far been unsuccessful with β-lactams (11, 17-20). In view of the known peptidoglycan activity in stationary phase and drug-specific differences for other metabolic approaches (6), an approach was conceived as disclosed herein to optimize a metabolic strategy for β-lactams.

Various expressly contemplated components of certain compositions and methods of the instant disclosure are considered in additional detail below.

### Metabolic Stimulants/Metabolites

In certain aspects, the compositions and methods of the instant disclosure provide a metabolic stimulant to bacteria that are or are at risk of becoming antibiotic tolerant. In some aspects, metabolic stimulants provide a carbon source to bacteria. Metabolic stimulants of the instant disclosure include, but are not limited to, sugars and their analogs, such as, glucose, mannitol, and fructose, and analogs thereof. For example, metabolites such as acetate, citrate, isocitrate, α-ketoglutarate, succinate, fumarate, malate and oxaloacetate. In some embodiments, metabolic stimulants for use in the compositions and methods described herein include, but are not limited to, alanine, cysteine, serine, valine, threonine, isoleucine, methionine, aspartate, tyrosine, phenylalanine, arginosuccinate, methylmalonic acid, propionic acid, acetoacetic acid, hydroxybutyrate, and analogs or derivatives thereof. Exemplary metabolic stimulants/carbon sources of the disclosure include: L-Arabinose, N-Acetyl-D-Glucosamine, D-Saccharic acid, Succinic acid, D-Galactose, L-Aspartic acid, L-Proline, D-Alanine, D-Trehalose, D-Mannose, Dulcitol, D-Serine, D-Sorbitol, Glycerol, L-Fucose, D-Glucuronic acid, D-Gluconic acid, DL-a-Glycerol Phosphate, D-Xylose, L-Lactic acid, Formic acid, D-Mannitol, L-Glutamic acid, D-Glucose-6-Phosphate, D-Galactonic acid-g-Lactone, D/L-Malic acid, D-Ribose, Tween 20, L-Rhamnose, D-Fructose, Acetic acid, a-D-Glucose, Maltose, D-Melibiose, Thymidine, L-Asparagine, D-Aspartic acid, D-Glucosaminic acid, 1,2-Propanediol, Tween 40, a-Ketoglutaric acid, a-Ketobutyric acid, a-Methyl-D-Galactoside, a-D-Lactose, Lactulose, Sucrose, Uridine, L-Glutamine, m-Tartaric acid, D-Glucose-1-Phosphate, D-Fructose-6-Phosphate, Tween 80, a-Hydroxyglutaric acid-g-Lactone, a-Hydroxybutyric acid, b-Methyl-D-Glucoside, Adonitol, Maltotriose, 2′-Deoxyadenosine, Adenosine, Gly-Asp, Citric acid, m-Inositol, D-Threonine, Fumaric acid, Bromosuccinic acid, Propionic acid, Mucic acid, Glycolic acid, Glyoxylic acid, D-Cellobiose, Inosine, Gly-Glu, Tricarballylic acid, L-Serine, L-Threonine, L-Alanine, Ala-Gly, Acetoacetic acid, N-Acetyl-D-Mannosamine, Mono-Methylsuccinate, Methylpyruvate, D-Malic acid, L-Malic acid, Gly-Pro, p-Hydroxyphenyl Acetic acid, m-Hydroxyphenyl Acetic acid, Tyramine, D-Psicose, L-Lyxose, Glucuronamide, Pyruvic acid, L-Galactonic acid-g-Lactone, D-Galacturonic acid, Phenylethylamine, 2-Aminoethanol, Chondroitin Sulfate C, a-Cyclodextrin, b-Cyclodextrin, g-Cyclodextrin, Dextrin, Gelatin, Glycogen, Inulin, Laminarin, Mannan, Pectin, N-Acetyl-D-Galactosamine, N-Acetyl-Neuraminic acid, b-D-Allose, Amygdalin, D-Arabinose, D-Arabitol, L-Arabitol, Arbutin, 2-Deoxy-D-Ribose, i-Erythritol, D-Fucose, 3-O-b-D-Galactopyranosyl-D-Arabinose, Gentiobiose, L-Glucose, D-Lactitol, D-Melezitose, Maltitol, a-Methyl-D-Glucoside, b-Methyl-D-Galactoside, 3-Methylglucose, b-Methyl-D-Glucuronic acid, a-Methyl-D-Mannoside, b-Methyl-D-Xyloside, Palatinose, D-Raffinose, Salicin, Sedoheptulosan, L-Sorbose, Stachyose, D-Tagatose, Turanose, Xylitol, N-Acetyl-D-Glucosaminitol, g-Amino-N-Butyric acid, d-Amino Valeric acid, Butyric acid, Capric acid, Caproic acid, Citraconic acid, Citramalic acid, D-Glucosamine, 2-Hydroxybenzoic acid, 4-Hydroxybenzoic acid, b-Hydroxybutyric acid, g-Hydroxybutyric acid, a-Keto-Valeric acid, Itaconic acid, 5-Keto-D-Gluconic acid, D-Lactic acid Methyl Ester, Malonic acid, Melibionic acid, Oxalic acid, Oxalomalic acid, Quinic acid, D-Ribono-1,4-Lactone, Sebacic acid, Sorbic acid, Succinamic acid, D-Tartaric acid, L-Tartaric acid, Acetamide, L-Alaninamide, N-Acetyl-L-Glutamic acid, L-Arginine, Glycine, L-Histidine, L-Homoserine, Hydroxy-L-Proline, L-Isoleucine, L-Leucine, L-Lysine, L-Methionine, L-Ornithine, L-Phenylalanine, L-Pyroglutamic acid, L-Valine, D,L-Carnitine, sec-Butylamine, D,L-Octopamine, Putrescine, Dihydroxyacetone, 2,3-Butanediol, 2,3-Butanedione and 3-Hydroxy-2-butanone. In certain embodiments, it is expressly contemplated that D-amino acids (whether naturally occurring or synthetic, including modified and/or derivative forms of such D-amino acids) can function as a metabolic stimulant.

### D-Amino Acids

In certain aspects, the present disclosure provides for use of non-canonical D-amino acids, optionally in combination with metabolic stimulants, to potentiate antibiotic (e.g beta-lactam antibiotic) sensitivity in bacteria, including tolerant stationary phase bacteria. Amino acids have a α-carbon that is connected to four functional groups: an amine group (—NH₂), a carboxyl group (—COOH), a hydrogen (—H) and a side chain (—R). Depending on the spatial arrangement of these four different groups, two stereoisomers exist: the levorotatory (L) and the dextrorotatory (D). These stereoisomers are not superimposable mirror images to each other. D-amino acids are also fundamental in microbial physiology where they are key constituents of the peptidoglycan (PG), an essential part of the bacterial cell wall. D-amino acids can also target distinctive cellular pathways in bacteria, and have been described to possess antibiofilm and bactericidal effect. In embodiments of the instant disclosure, application of D-amino acids is an attractive antimicrobial strategy, either alone or in synergy with existing antibiotics. Moreover, it has previously been demonstrated that combinatory treatments that include several D-amino acids can be more effective than individual D-amino acids and prevent the emergence of suppressor mutants, since, without wishing to be bound by theory, different D-amino acids are believed to target distinct pathways.

Naturally occurring D-Amino acids include D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-asparagine, and D-tyrosine. As an example, D-alanine has the following structure:

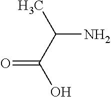

Synthetic D-Amino acids (e.g. D-phenylglycine, D-norleucine, etc.), which tend to constitute modified forms of the naturally occurring amino acids, are also expressly contemplated for use in certain aspects of the instant disclosure. Exemplary synthetic D-amino acids include but are not limited to D-phenylglycine, D-alpha-aminobutyric acid, and D-alpha-aminopimelic acid, which have the following structures:

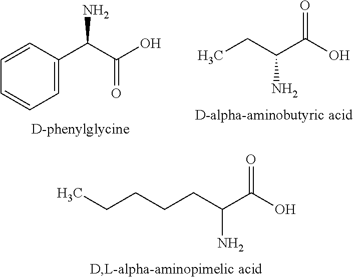

Antibiotics that Induce Tolerance in Bacteria

Certain aspects of the present disclosure relate to compositions and methods that either include antibiotics to which bacteria are at risk of developing tolerance, and/or that potentiate the antibacterial effects of antibiotics to which bacteria develop tolerance or are at risk of developing tolerance. In certain aspects, the antibiotics of the instant disclosure include β-lactam antibiotics, aminoglycoside antibiotics, quinolone antibiotics, and/or carbapenem antibiotics (e.g., imipenem). In some aspects, the antibiotics of the instant disclosure include β-lactams, carbapenems (e.g., imipenem), aminoglycosides, fluoroquinolones, related quinolones and naphthyridines, chloramphenicol, macrolides, ketolides, azalides, Synercid®, tetracyclines, glycopeptides, novobiocin, oxazolidinones, cephalosporins, ceftazidime, ciprofloxacin, gentamicin, meropenem and the like, or a combination thereof. In certain embodiments, an exemplary antibiotic of a composition and/or method of the instant disclosure and/or that is potentiated via the compositions and/or methods of the instant disclosure is an aminoglycoside antibiotic (e.g., gentamicin, streptomycin, kanamycin), a β-lactam antibiotic (e.g., penicillins and cephalosporins), a vancomycin antibiotic, a bacitracin antibiotic, a macrolide antibiotic (e.g., erythromycins), a lincosamide antibiotic (e.g., clindomycin), a chloramphenicol antibiotic, a tetracycline antibiotic, an amphotericin antibiotic, a cefazolin antibiotic, a clindamycin antibiotic, a mupirocin antibiotic, a sulfonamide antibiotic, a trimethoprim antibiotic, a rifampicin antibiotic, a metronidazole antibiotic, a quinolone antibiotic, a novobiocin antibiotic, a polymixin antibiotic, a gramicidin antibiotic, alone or in combination, or any salts or variants thereof.

### β-Lactam Antibiotics

Certain aspects of the instant disclosure employ β-lactam antibiotics. β-lactam antibiotics are a class of broad-spectrum antibiotic that consists of antibiotic agents that contain a beta-lactam ring in their molecular structures. Exemplary β-lactam antibiotics include the following:
Penicillin derivatives (Penams), for which an exemplary dosage is a standard adult dosage between 0.2-1.0 g in a 6-24 hour interval: Specific examples include Benzathine penicillin (benzathine & benzylpenicillin), Benzylpenicillin (penicillin G), Phenoxymethylpenicillin (penicillin V), Procaine penicillin (procaine & benzylpenicillin), Pheneticillin, Cloxacillin, Dicloxacillin, Flucloxacillin, Methicillin, Nafcillin, Oxacillin, Temocillin, Amoxicillin, Ampicillin, Mecillinam, Carbenicillin, Ticarcillin, Azlocillin, Mezlocillin, and Piperacillin, with exemplary structures as shown below.

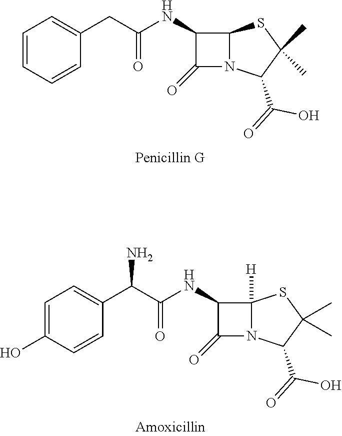

Cephalosporin (Cephems), for which an exemplary dosage is a standard adult dosage between 0.2-1.0 g in a 6-24 hour interval: Examples include Cefazolin, Cephalexin, Cephalosporin C, Cephalothin, Cefaclor, Cefamandole, Cefuroxime, Cefotetan, Cefoxitin, Cefixime, Cefotaxime, Cefpodoxime, Ceftazidime, Ceftriaxone, Cefepime, Cefpirome, Cefsulodin and Ceftaroline. Exemplary structures for such compounds include the following.

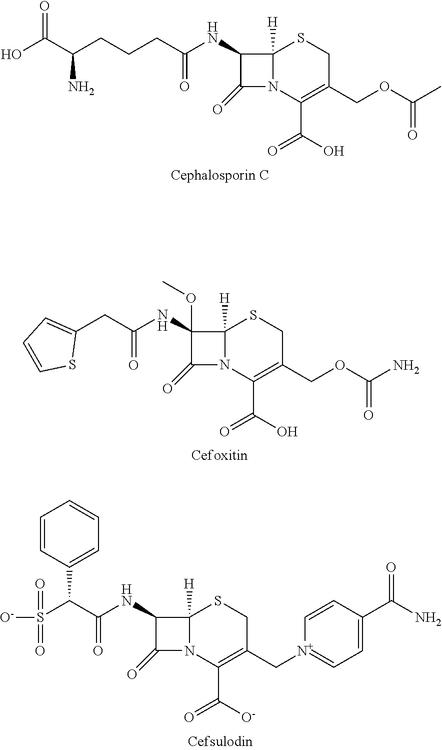 [SMILES: CC(=O)OCC1=C(C(=O)O)N2C(=O)[C@@H](NC(=O)CCC[C@@H](N)C(=O)O)[C@H]2SC1 ; CO[C@@]1(NC(=O)Cc2cccs2)C(=O)N2C(C(=O)O)=C(COC(N)=O)CS[C@@H]21 ; NC(=O)c1cc[n+](CC2=C(C(=O)[O-])N3C(=O)[C@@H](NC(=O)[C@@H](c4ccccc4)S(=O)(=O)[O-])[C@H]3SC2)cc1]

Monobactams, for which an exemplary dosage is a standard adult dosage between 0.5-2.0 g in a 6-12 hour interval: Examples include Aztreonam, Tigemonam, Nocardicin A, and Tabtoxinine β-lactam (which does not inhibit penicillin-binding proteins). An exemplary structure for such compounds follows.

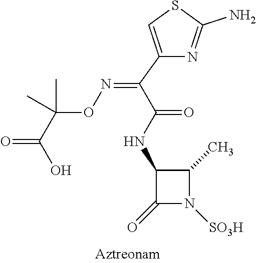 [SMILES: C[C@H]1[C@H](NC(=O)/C(=N\OC(C)(C)C(=O)O)c2csc(N)n2)C(=O)N1S(=O)(=O)O]

Carbapenems and Penems, for which an exemplary dosage is a standard dosage between 0.5-2.0 g in a 8 hour interval: Examples include Biapenem, Doripenem, Ertapenem, Faropenem, Imipenem, Meropenem, Panipenem, Razupenem, Tebipenem, and Thienamycin. An exemplary structure for such compounds follows.

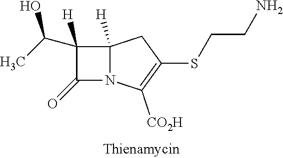 [SMILES: C[C@@H](O)[C@H]1C(=O)N2C(C(=O)O)=C(SCCN)C[C@H]12]

Most β-lactam antibiotics are believed to work by inhibiting cell wall biosynthesis in the bacterial organism. β-lactam antibiotics are the most widely used group of antibiotics. Until 2003, when measured by sales, more than half of all commercially available antibiotics in use were β-lactam compounds (Slander, R. P. Applied Microbiology and Biotechnology. 61: 385-392). β-lactam antibiotics are indicated for the prevention and treatment of bacterial infections caused by susceptible organisms. At first, β-lactam antibiotics were mainly active only against Gram-positive bacteria, yet the recent development of broad-spectrum β-lactam antibiotics active against various Gram-negative organisms has increased their usefulness.

### β-Lactamase Inhibitors

Certain aspects of the instant disclosure include and/or employ β-lactamase inhibitors. In certain embodiments, β-lactamase inhibitors can be compounded together with antibiotics to potentiate antibiotic efficacy. β-lactamases are a family of enzymes involved in bacterial resistance to β-lactam antibiotics. Without wishing to be bound by theory, β-lactamases tend to act by breaking the beta-lactam ring that is believed to be necessary for the antimicrobial activity of penicillin-like antibiotics. With the goal of enhancing the activity of penicillin-like compounds in the presence of β-lactamases, β-lactamase inhibitors have been developed (Essack SY. Pharmaceutical Research. 18: 1391-9). Although β-lactamase inhibitors possess little antibiotic activity of their own (“Beta-Lactamase Inhibitors”. Department of Nursing of the Fort Hays State University College of Health and Life Sciences. October 2000), they prevent bacterial degradation of beta-lactam antibiotics and can extend the range of bacteria such drugs are effective against.

β-lactamase inhibitors are generally co-formulated with a β-lactam antibiotic possessing a similar serum half-life. This is done to minimize resistance development that might occur as a result of varying exposure to one or the other drug. The main classes of β-lactam antibiotics used to treat gram-negative bacterial infections include penicillins, 3rd generation cephalosporins (e.g., cefsulodin), and carbapenems. β-lactamase inhibitors expand the useful spectrum of these β-lactam antibiotics by inhibiting the β-lactamase enzymes produced by bacteria to deactivate them (Watson et al. Clinical Pharmacokinetics. 15: 133-64).

Exemplary β-lactamase inhibitors possessing a β-lactam core include:
Tebipenem, which was the first carbapenem to be administered orally in the form of Tebipenem-Pivoxil.
Boron based transition state inhibitors (“BATSIs”), which constitute a very potent group of beta-lactamase inhibitors. An exemplary BATSI is Ec19.
Clavulanic acid or clavulanate, which is often combined with amoxicillin (Augmentin) or ticarcillin (Timentin). Clavulanic Acid has the following exemplary structure:

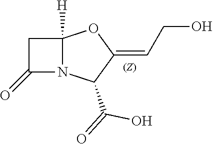 [SMILES: O=C(O)[C@H]1/C(=C/CO)O[C@@H]2CC(=O)N21]

Sulbactam, which is often combined with ampicillin (Unasyn) or Cefoperazone (Sulperazon). Sulbactam has the following structure:

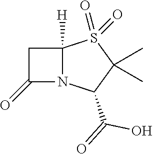

Tazobactam, which is often combined with piperacillin (Zosyn) (Tazocin). Tazobactam has the following structure:

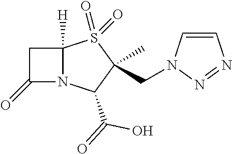 [SMILES: C[C@]1(Cn2ccnn2)[C@H](C(=O)O)N2C(=O)C[C@H]2S1(=O)=O]

Exemplary non-β-lactam β-lactamase inhibitors include:
Avibactam, which has been FDA approved in combination with ceftazidime (Avycaz), and is currently undergoing clinical trials for combination with ceftaroline. Avibactam has the following structure:

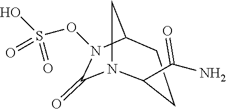 [SMILES: NC(=O)C1CCC2CN1C(=O)N2OS(=O)(=O)O]

Relebactam (previously known as MK-7655) is undergoing Phase III clinical trials as a treatment for pneumonia and bacterial infections. Relebactam has the following structure:

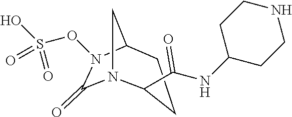 [SMILES: O=C(NC1CCNCC1)C1CCC2CN1C(=O)N2OS(=O)(=O)O]

### Antibiotic Tolerant Bacteria

In certain aspects, the present disclosure provides compositions and/or methods designed to inhibit the growth of and/or kill bacteria, particularly bacteria that have become antibiotic tolerant and/or antibiotic resistant. Tolerance specifically refers to an inability of high concentrations of antibiotics—typically lethal concentrations that are above the growth-inhibitory threshold for a given strain—to kill bacteria. Tolerance levels can be influenced by genetic mutations or induced by environmental conditions. Bacteria can often develop antibiotic tolerance and/or resistance. Resistance can tend to arise via mutations that confer increased survival, which are selected for in natural selection, and which can arise quickly in bacteria because lifespans and production of new generations can be on a timescale of mere hours. Tolerant and/or resistant microbes are more difficult to treat, requiring alternative medications or higher doses of antimicrobials. These approaches may be more expensive, more toxic or both. Microbes resistant to multiple antimicrobials are called multidrug resistant (MDR). Those considered extensively drug resistant (XDR) or totally drug resistant (TDR) are sometimes called “superbugs”.

An exemplary list of Gram-positive bacteria that have been shown to possess antibiotic resistance or have been associated with persistent bacterial infections includes Clostridium difficile, Enterococcus, Mycobacterium tuberculosis, Mycobacterium avium complex (including Mycobacterium intracellulare and Mycobacterium avium), Mycobacterium smegmatis, Mycoplasms genitalium, Staphylococcus aureus, Streptococcus pyogenes, Streptococcus pneumoniae, and Mycobaterium leprae.

An exemplary list of Gram-negative bacteria that have been shown to possess antibiotic resistance or have been associated with persistent bacterial infections includes Campylobacter, Neisseria gonorrhoeae, Enterobacteriaceae, Klebsiella pneumoniae, Salmonella, Escherichia coli, Acinetobacter, Pseudomonas aeruginosa, Burkholderia pseudomallei, Burkholderia cenocepacia, Helicobacter pylori, and Hemophilus influenza. (See, e.g., Cohen et al. Cell Host & Microbe 13: 632-642, the contents of which are incorporated by reference herein in their entirety.)

Treponema pallidum has also been described as associated with persistent bacterial infections (see Grant and Hung. Virulence 4: 273-283, the contents of which are incorporated by reference herein in their entirety).

The instant disclosure expressly contemplates targeting of any of (or any combination of) the above-listed forms of Gram-positive and/or Gram-negative bacteria, particularly those forms of the above-recited bacteria that possess or are at risk of developing antibiotic tolerance and/or antibiotic resistance.

### Methods of Treatment

The compositions and methods of the present disclosure may be used in the context of a number of therapeutic or prophylactic applications. Compositions of the instant disclosure can be selected and/or administered as a single agent, or to augment the efficacy of another therapy (second therapy), it may be desirable to combine these compositions and methods with one another, or with other agents and methods effective in the treatment, amelioration, or prevention of diseases and/or infections.

In certain embodiments of the instant disclosure, one or more metabolic stimulants, one or more D-amino acids and optionally one or more β-lactamase inhibitors can be administered to a subject, optionally together with administration of an antibiotic for which activity enhancement is desired.

Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

### Pharmaceutical Compositions

Agents of the present disclosure can be incorporated into a variety of formulations for therapeutic use (e.g., by administration) or in the manufacture of a medicament (e.g., for treating or preventing a bacterial infection) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers or diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethyl succinates.

Furthermore, the term “pharmaceutically acceptable prodrugs” as used herein refers to those prodrugs of certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent, such as a metabolic stimulant, a D-amino acid and/or a β-lactamase inhibitor, optionally together with an antibiotic, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease/infection to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein may be used (e.g., administered to an individual, such as a human individual, in need of treatment with a metabolic stimulant, a D-amino acid and/or a β-lactamase inhibitor, optionally together with an antibiotic) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, German® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such as magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., a metabolic stimulant, a D-amino acid and/or a β-lactamase inhibitor, optionally together with an antibiotic) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Drugs provided herein can be formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for oral delivery or intravenous injection to a subject.

The exact amount of an agent required to achieve an effective amount will vary from subject to subject, depending, for example, on species, age, and general condition of a subject, severity of the side effects or disorder/infection, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a metabolic stimulant, a D-amino acid and/or a (3-lactamase inhibitor, optionally together with an antibiotic) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

A drug or other therapy of the instant disclosure is administered to the subject in an amount sufficient to achieve a desired effect at a desired site (e.g., reduction of bacterial infection, bacterial abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue, the frequency of administering the multiple doses to the subject or applying the multiple doses to the tissue is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a metabolic stimulant, a D-amino acid and/or a β-lactamase inhibitor, optionally together with an antibiotic y) described herein. In certain embodiments, a dose described herein includes independently between 1 mg and 3 mg, inclusive, of an agent (e.g., a metabolic stimulant, a D-amino acid and/or a β-lactamase inhibitor, optionally together with an antibiotic) described herein. In certain embodiments, a dose described herein includes independently between 3 mg and 10 mg, inclusive, of an agent (e.g., a metabolic stimulant, a D-amino acid and/or a β-lactamase inhibitor, optionally together with an antibiotic) described herein. In certain embodiments, a dose described herein includes independently between 10 mg and 30 mg, inclusive, of an agent (e.g., a metabolic stimulant, a D-amino acid and/or a β-lactamase inhibitor, optionally together with an antibiotic) described herein. In certain embodiments, a dose described herein includes independently between 30 mg and 100 mg, inclusive, of an agent (e.g., a metabolic stimulant, a D-amino acid and/or a β-lactamase inhibitor, optionally together with an antibiotic) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., a metabolic stimulant, a D-amino acid and/or a β-lactamase inhibitor, optionally together with an antibiotic) or composition, as described herein, can be administered in combination with one or more additional pharmaceutical agents (e.g., therapeutically and/or prophylactically active agents), which are different from the agent or composition and may be useful as, e.g., combination therapies.

The agents or compositions can be administered in combination with additional pharmaceutical agents that improve their activity (e.g., activity (e.g., potency and/or efficacy) in treating a disease or infection (e.g., an antibiotic tolerant or resistant bacterial infection) in a subject in need thereof, in preventing a disease or infection in a subject in need thereof, in reducing the risk of developing a disease or infection in a subject in need thereof, etc. in a subject or tissue. In certain embodiments, a pharmaceutical composition described herein including an agent (e.g., a metabolic stimulant, a D-amino acid and/or a β-lactamase inhibitor, optionally together with an antibiotic) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an immunotherapy, an antioxidant, an antiinflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or infection described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, additional antibiotics, antimicrobials, anti-proliferative agents, cytotoxic agents, anti-angiogenesis agents, antiinflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising an agent (e.g., a D-amino acid, optionally with a metabolic stimulant) and/or composition (e.g., a metabolic stimulant, a D-amino acid and/or a β-lactamase inhibitor, optionally together with an antibiotic) of this disclosure. In some embodiments, the kits further include instructions for use in accordance with the methods of this disclosure. In some embodiments, these instructions comprise a description of administration of the agent to treat or prevent, e.g., an infection and/or disease. In some embodiments, the instructions comprise a description of how to administer a D-amino acid, a carbon source and/or an antibiotic to a bacterial population, a subject infected or suspected to be infected or at risk of infection with a bacteria.

The instructions generally include information as to dosage, dosing schedule, and route of administration for the intended use/treatment. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Reagents

Antibiotics, most carbon sources, amino acids, and PBS were purchased from Sigma-Aldrich™ except for glucose which was purchased from Fisher Scientific. LB Broth and 7H9 medium were purchased from Difco™. MOPS media was purchased from Teknova™.

### Bacterial Strains

All experiments unless otherwise noted used Escherichia coli K12 strain MG1655. Where noted, other strains used for this study include E. coli BW25113 and M. smegmatis mc²155. The clinical isolates of Escherichia coli (isolate DICON-146) and Klebsiella pneumoniae (isolate DICON-135) were from Duke Medical Center. E. coli genetic knockout ΔdadA was constructed by P1 transduction from the Keio collection into MG1655. Colony PCR was used to verify deletion accuracy.

### Culture Conditions

E. coli and K. pneumoniae were grown in LB medium. Where noted, E. coli was grown in MOPS EZ Rich Medium with 10 mM glucose. Overnight cultures of E. coli or K. pneumoniae were inoculated from glycerol stocks and grown in non-baffled flasks for 24 hours at 37° C. shaking at 300 RPM. Overnight cultures were grown in the final treatment medium (1% LB diluted in PBS unless otherwise noted as 100% LB, 10% LB in PBS, or 1% MOPS Rich in PBS). M. smegmatis was grown in Middlebrook 7H9 supplemented with 0.05% oleic acid, 2% dextrose, and 0.004% catalase (OADC), 0.2% glycerol, and 0.05% tyloxapol. Overnight cultures of M. smegmatis were inoculated from glycerol stocks into undiluted 7H9 medium and grown in non-baffled flasks for 48 hours at 37° C. shaking at 300 RPM. Overnight cultures of M. smegmatis were then diluted 1:100 in PBS with 0.05% tyloxapol prior to antibiotic treatment.

### Killing Assays

Overnight cultures were dispensed into round bottom 96 well plates with appropriate amounts of antibiotic and supplemented metabolites. All metabolites were used at 10 mM unless otherwise noted. Plates were incubated for 24 hours (E. coli and K. pneumoniae) or 48 hours (M. smegmatis) at 37° C., 900 RPM, and 60% humidity. Plates were washed once in PBS before being diluted in PBS and plated on LB for CFU quantification. M. smegmatis was washed and diluted in PBS with 0.05% tyloxapol. Data is presented by the mean with error bars given by standard deviation. For experiments that exhibited day-to-day variability, additional replicates were completed, and data are presented by the mean (thick lines) with the range given by the shaded region and individual replicates shown as thin lines.

### OD Measurements

Overnight cultures were dispensed into round bottom 96 well plates with appropriate amounts of antibiotic and supplemented metabolites. All metabolites were used at 10 mM unless otherwise noted. Plates were incubated for 24 hours at 37° C., 900 RPM, and 60% humidity. Optical density was measured at 600 nm on a SpectraMax M3 Microplate Reader spectrophotometer (Molecular Devices™). Description and quantification of growth (biomass accumulation, change in OD) are from samples with no antibiotic added.

### MIC Determination

The minimum inhibitory concentration for a given antibiotic and metabolite was defined by using a control culture that was not treated with metabolite to define a baseline, stationary phase optical density. For a metabolite-treated culture, the optical density dose-response for a given antibiotic was fit to a four parameter logistic curve, and the resulting parameters used to identify the antibiotic concentration corresponding to the optical density of the no metabolite control culture which corresponds to no change in optical density, or no growth or lysis of the culture. This concentration is denoted as the MIC. For measurements of MIC fold change, comparisons were made within the same treatment day. Curve fitting and calculations were done in Matlab™ R2018a (Mathworks™).

### CFU Growth Assays

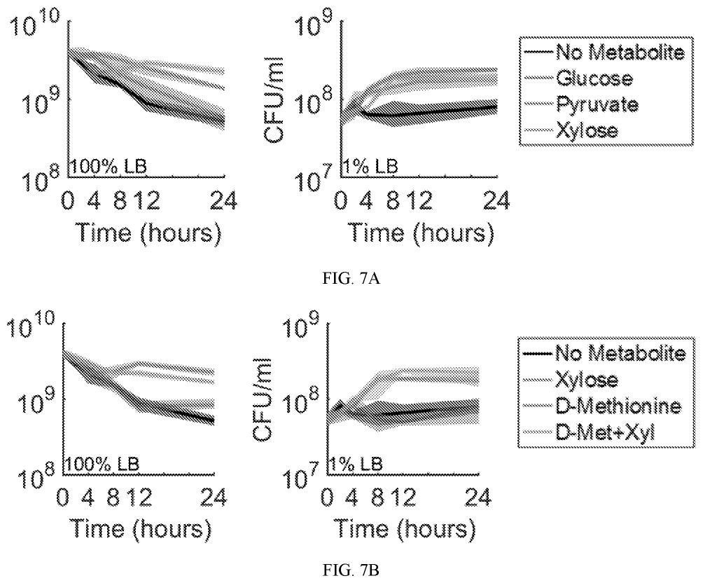

Overnight cultures were dispensed in volumes of 1 ml into 14 ml Falcon tubes with appropriate metabolites added at 10 mM. At 2, 4, 8, 12, and 24 hours, 10 μl of each culture was sampled directly into 90 μl PBS, serially diluted, and plated in duplicate in order to increase precision of CFU measurements, as shown in FIG. 7. Duplicate CFU measurements from individual experiments were averaged to obtain the final CFU measurement for that experiment. Data are presented by the mean (thick lines) of three biological replicates with the range given by the shaded region.

### Example 2: Metabolic Stimulation Sensitized Tolerant Stationary Phase Bacteria to Ampicillin and Other β-Lactam Antibiotics

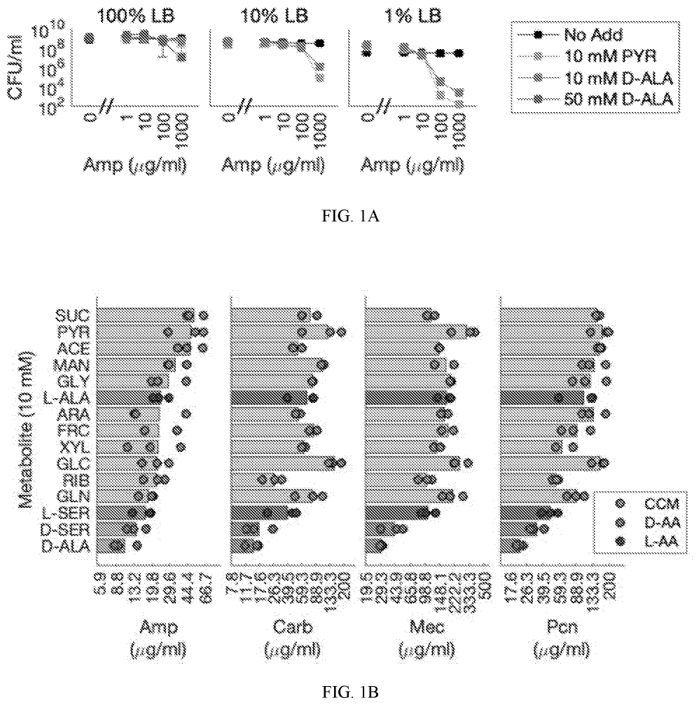

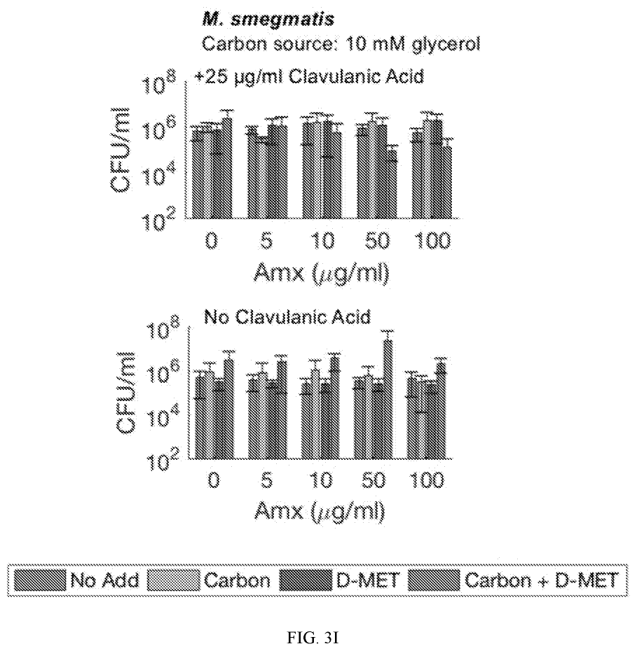

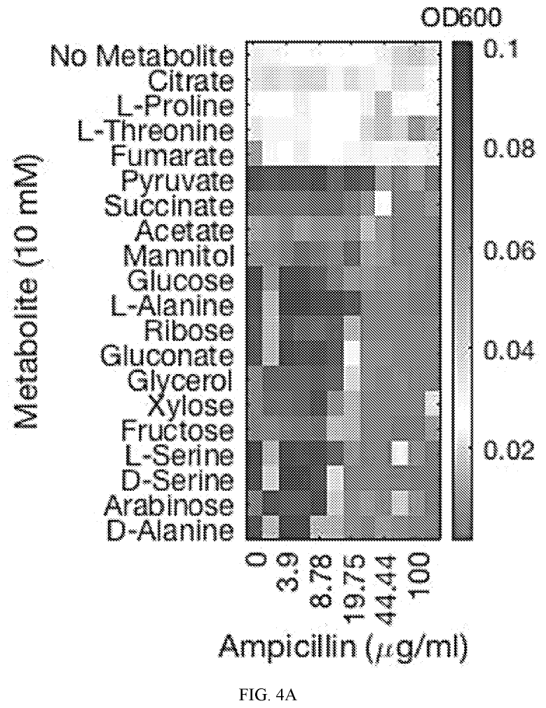

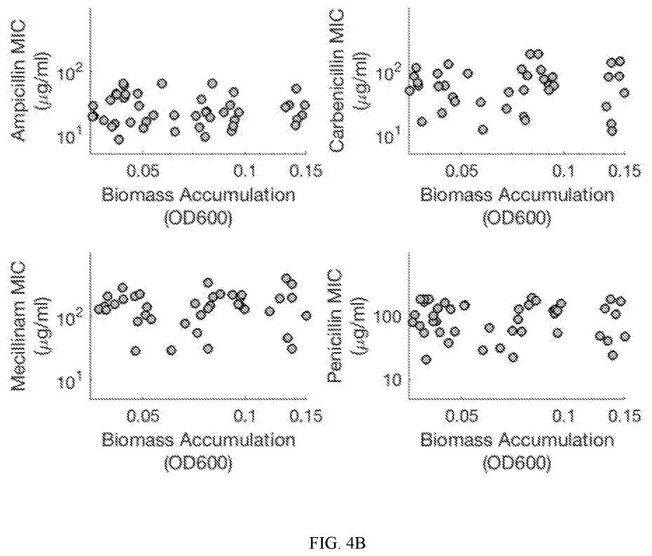

Tolerant bacteria were generated by growing wild-type Escherichia coli in LB medium for 24 hours. These stationary phase cultures were not killed by 24 hour treatment with 1 mg/ml ampicillin (FIG. 1A). Previous metabolism-directed strategies targeting antibiotic tolerance had proven unsuccessful with up to 100 μg/ml ampicillin (11, 19, 20), potentially reflecting the need to stimulate an at least 10-fold change in sensitivity. It was additionally observed that tolerance was not due to culture density, as cultures grown in LB diluted in PBS to reduce the stationary phase carrying capacity were equally tolerant to ampicillin. Thus, β-lactam tolerance in stationary phase was observed to be nutrient-dependent, not density-dependent. Reasoning that lower density cultures would likely be more responsive to metabolic stimulation, 1% LB was used to screen for metabolites able to sensitize cultures to ampicillin, as measured by optical lysis (FIG. 4A). Colony forming units (CFUs) of cultures treated with ampicillin and either D-alanine or pyruvate, representative of strong and weak performing metabolites, were enumerated, and the results obtained confirmed that the observed optical readings reflected cell death (FIG. 1A). As expected, sensitizing high density cultures required higher drug and metabolite concentrations than had been tried previously in the art (11, 19, 20). These results demonstrated that metabolic stimulation was indeed able to sensitize tolerant stationary phase bacteria to the β-lactam ampicillin.

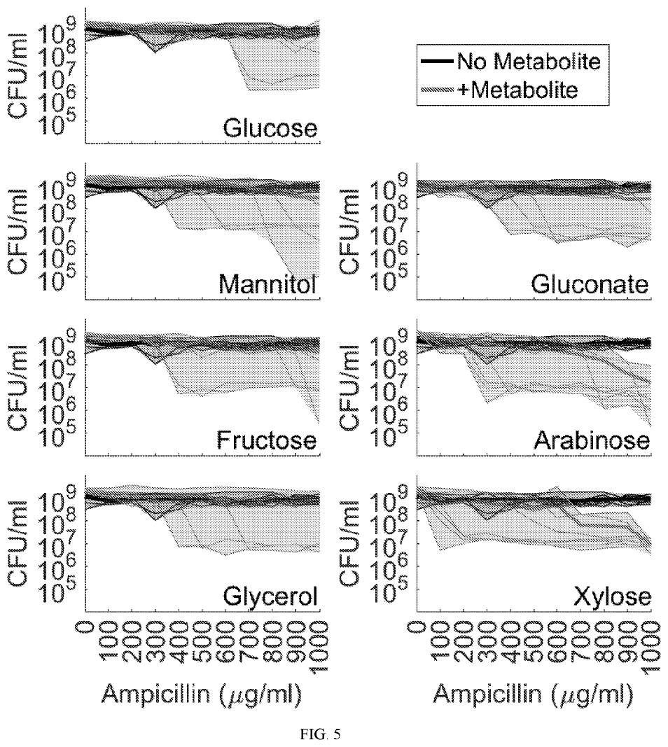

To compare the efficacy of supplementation with different metabolites across a range of β-lactam antibiotic treatments, metabolite-enabled minimal inhibitory concentrations (MICs) were quantified for β-lactams from four different generations of penicillins ampicillin, carbenicillin, mecillinam, and penicillin (FIG. 1B and FIG. 4B). The relative potency of each metabolite tested was observed to be similar for each of the four different β-lactams tested. Additionally, while different metabolites stimulated up to six-fold increases in optical density, measured MICs for each drug were not proportional to metabolite-stimulated biomass accumulation, which indicated that differences in potentiation were not solely due to differences in growth stimulation by each metabolite (FIG. 4B). It was also noted that similar results were observed for experiments performed in 100% LB, which indicated that the ability of a carbon source to potentiate beta-lactam killing was not explained by carbon sources stimulating an increase in culture density (FIGS. 5 and 7A).

Specific metabolic fluxes stimulated by individual metabolites have been previously described to tune aminoglycoside sensitivity (18); and it is contemplated that further comparison of fluxes stimulated by central carbon metabolites can similarly optimize the currently disclosed use of metabolite stimulation to sensitize tolerant bacteria to β-lactam antibiotics.

### Example 3: D-Amino Acid Supplementation Potentiated Sensitization of Tolerant Bacteria to β-Lactam Antibiotics

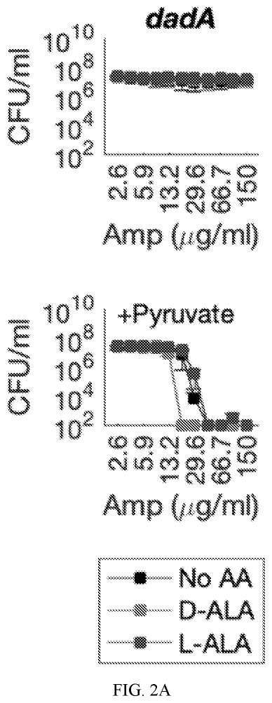

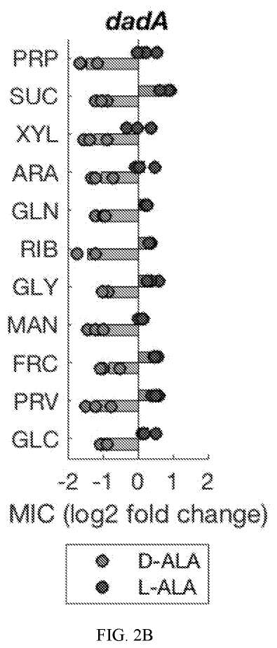

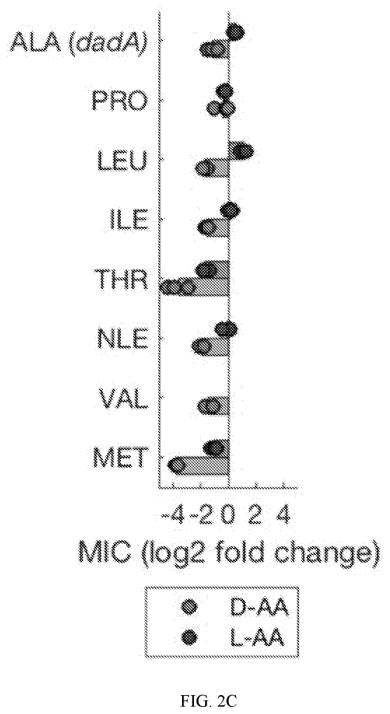

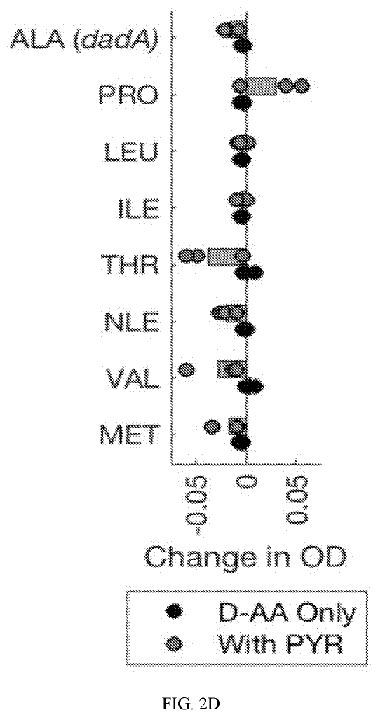

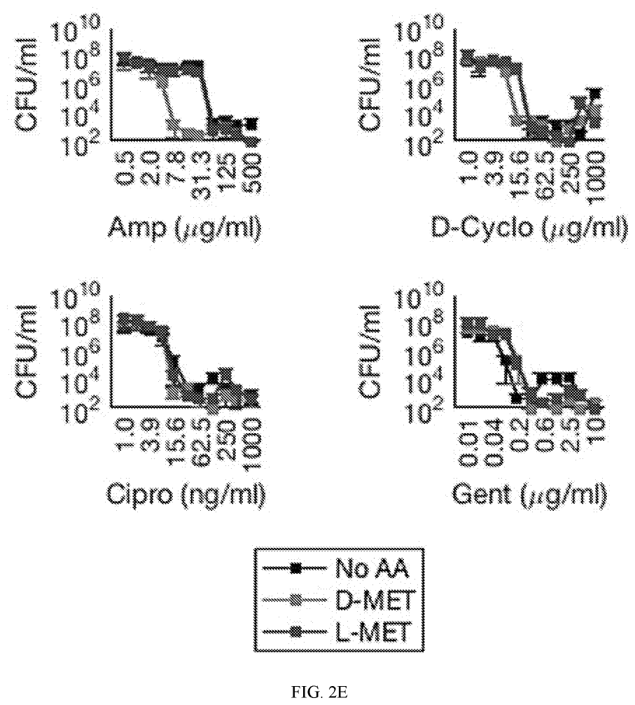

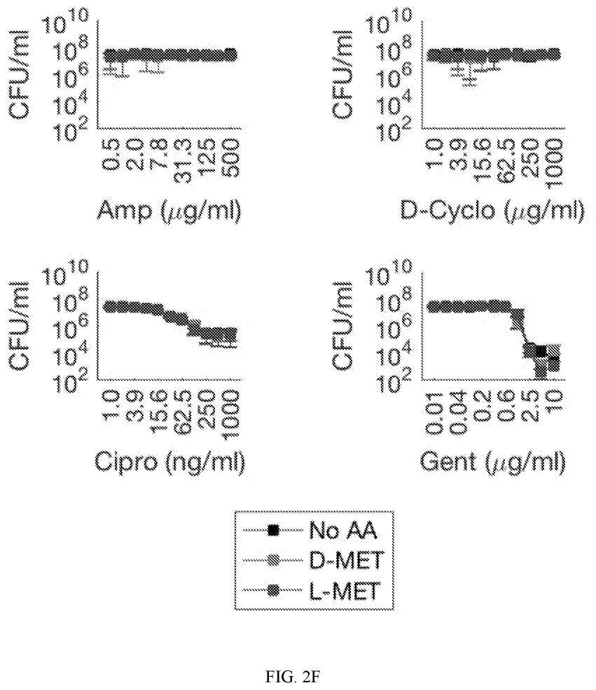

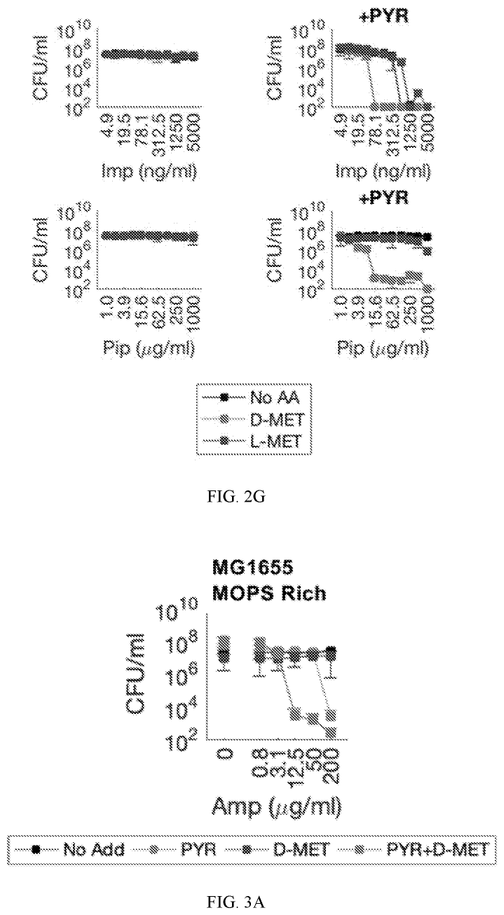

In the above example, supplementation with D-alanine or D-serine consistently led to the lowest observed MICs, and both D-isomers outperformed their corresponding L-isomers (FIG. 1B). Such effects likely reflected D-isomer specific effects upon bacterial peptidoglycans, rather than differences in stimulating central carbon metabolism, as both alanine and serine species are converted into pyruvate. To test this, the ability of D- and L-alanine to potentiate ampicillin sensitivity was examined in a genetic knockout strain (ΔdadA) that is unable to catabolize D-alanine into pyruvate. While D- and L-serine are catabolized by different enzymes, L-alanine is converted by DadX into D-alanine before being catabolized by DadA. Neither alanine isomer alone sensitized ΔdadA cultures to ampicillin (FIG. 2A). However, when the AdadA strain was complemented by supplementing with pyruvate, D-alanine was then identified to potentiate the efficacy of lower ampicillin concentrations. This observed potentiation by D-alanine was independent of the growth-supporting metabolite used to initially sensitize the culture (FIG. 2B), as D-alanine consistently lowered the ampicillin MIC across all tested metabolites, while L-alanine did not. These results confirmed that D-alanine influenced β-lactam lethality through additional processes outside of central carbon metabolism.

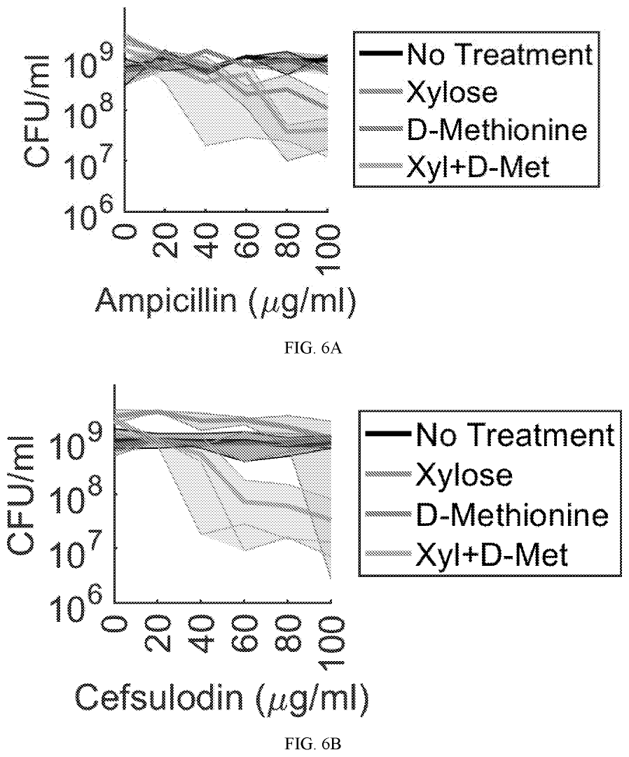

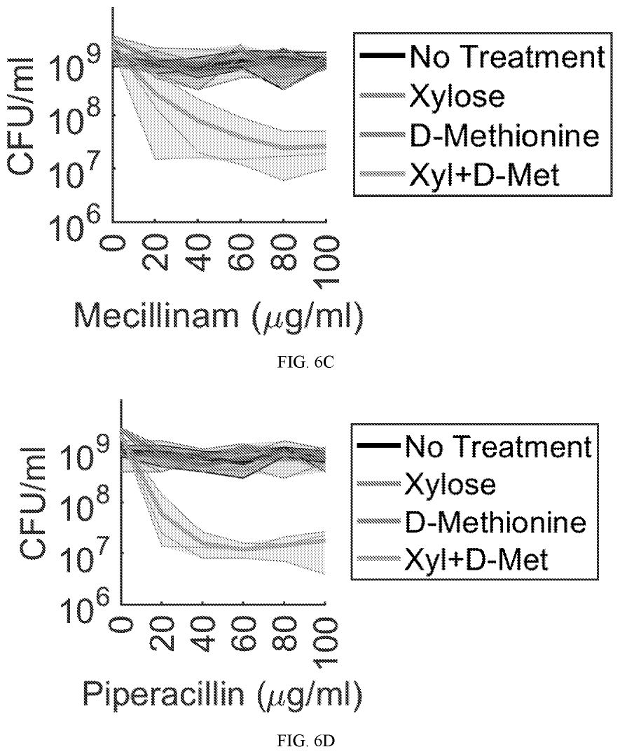

To determine if other D-amino acids could also potentiate β-lactams in these conditions, cultures were sensitized with pyruvate and changes in ampicillin MIC upon supplementation with seven other amino acid isomer pairs were then examined (FIG. 2C). All D-amino acids tested, except for D-proline, outperformed their corresponding L-isomer. Notably, previous work has shown that exogenous D-methionine, D-valine, and D-norleucine are incorporated into E. coli peptidoglycan, whereas D-proline is not (14). Potentiation by D-amino acids did not proceed through additional metabolic stimulation and growth as measured by optical density (OD, herein measured as a proxy for biomass) (FIG. 2D), and was specific to β-lactam antibiotics (FIGS. 2E, 2F, 2G, and 6). Collectively, and without wishing to be bound by theory, these results indicated that the currently observed potentiation of β-lactams by D-amino acids was due to specific perturbations to peptidoglycan structure.

### Example 4: The Effects of D-Amino Acid Supplementation were Generalizable to Various Forms of Tolerant Bacteria, Including β-Lactamase-Expressing E. coli, K. pneumoniae and Mycobacteria

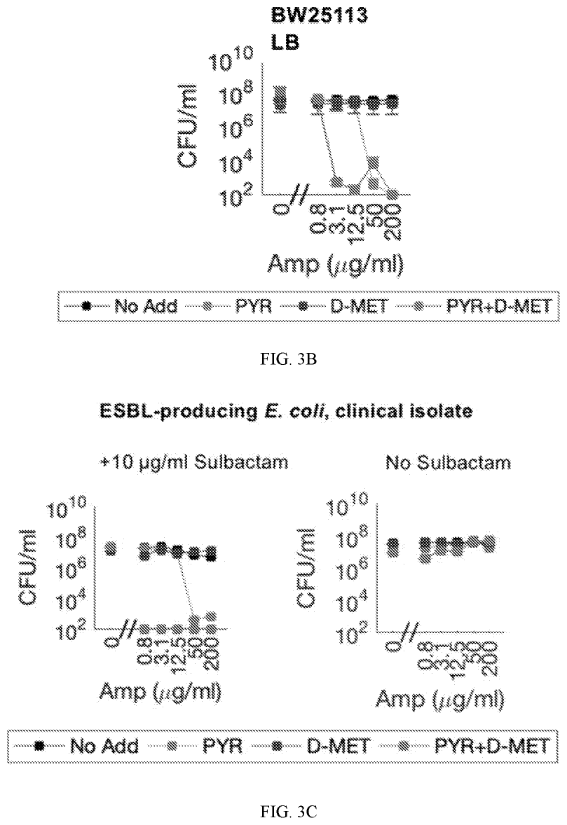

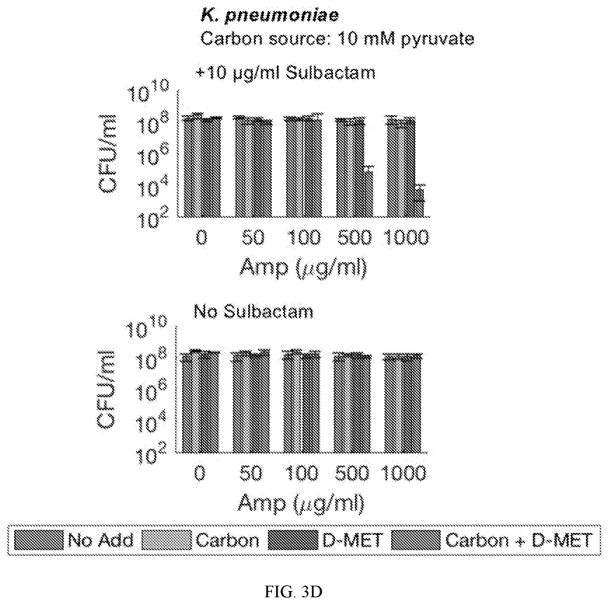

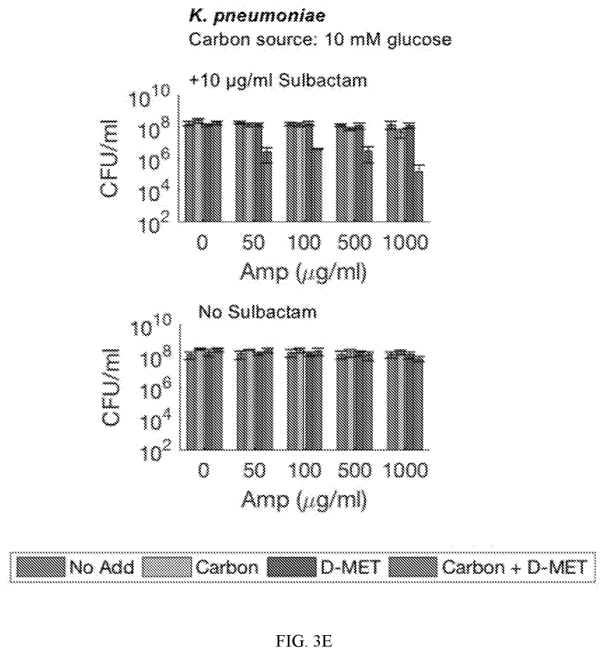

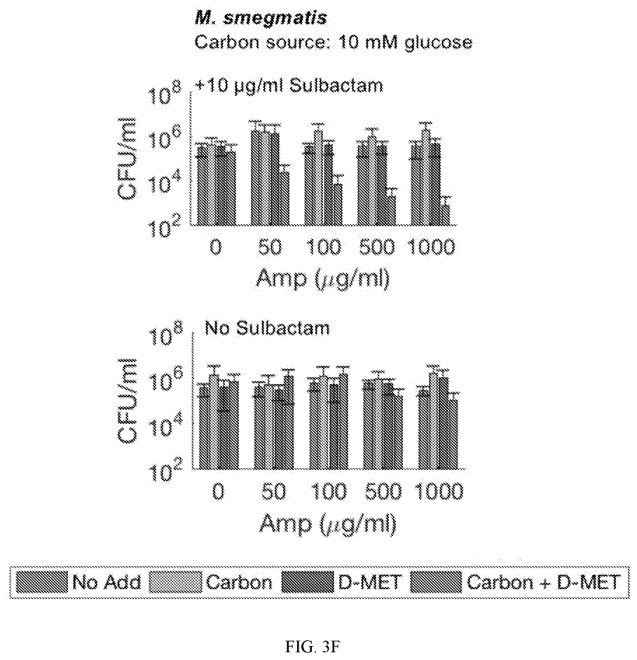

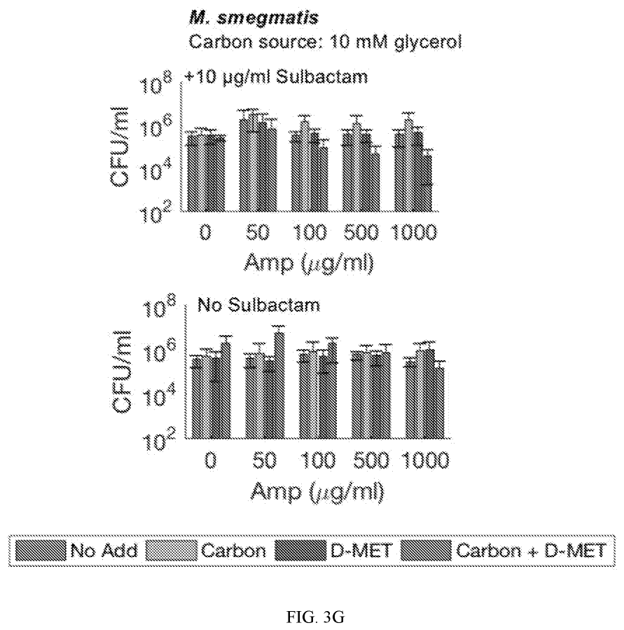

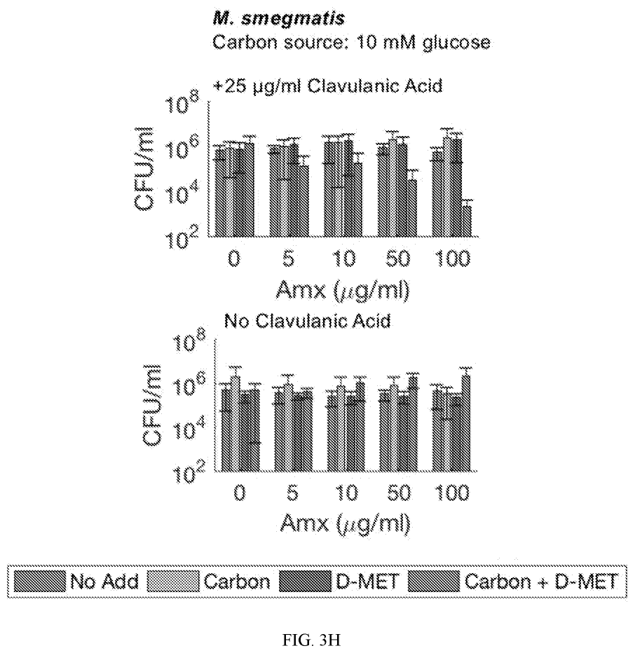

Having developed an anti-tolerance strategy for β-lactam antibiotics, the generalizability of the current findings was then assessed. First, D-methionine was chosen as the carbon source for such generalizability experiments because the observed effect size for D-methionine was larger than for D-alanine (FIG. 1B, FIG. 2C), which would therefore allow for better separation of initial metabolic sensitization from the additional D-amino acid-mediated potentiation effect. To test for media dependence, overnight cultures of E. coli MG1655 were grown in a defined rich medium, MOPS-rich medium. Supplementation with pyruvate restored ampicillin lethality against stationary phase cultures of MG1655 in MOPS-rich medium, and D-methionine further potentiated ampicillin (FIG. 3A). Pyruvate and D-methionine in combination similarly sensitized a different E. coli strain, BW25113, grown in LB medium (FIG. 3B). Whereas recent studies investigating NCDAA-induced biofilm disassembly have identified strain- and medium-dependent effects (21, 22), the current generalizability results demonstrated that the instant findings related to β-lactam sensitivity were not dependent on specific factors in LB medium or potential mutations in the tested MG1655 strain.

To determine if the currently disclosed findings generalized to resistant clinical isolates, tolerant, stationary phase cultures of a clinical isolate of E. coli, which is resistant to ampicillin due to a plasmid-borne extended spectrum β-lactamase (ESBL), were generated. ESBL enzymes can be inhibited by β-lactamase inhibitors such as sulbactam, which restores β-lactam sensitivity for exponentially growing bacteria (3). However, in tolerant cultures, sulbactam alone was unable to restore sensitivity to ampicillin (FIG. 3C). Combining sulbactam with pyruvate successfully restored sensitivity to cultures of resistant E. coli in stationary phase, and D-methionine further potentiated killing by lower drug concentrations (FIG. 3C). The current approach was also found to generalize to a resistant clinical isolate of the gram-negative bacterium Klebsiella pneumoniae, as the combination of all three components—pyruvate, D-methionine, and sulbactam—sensitized stationary phase cultures of a β-lactamase-producing K. pneumoniae isolate to ampicillin (FIG. 3D). As expected, pyruvate and D-methionine alone were unable to restore sensitivity to either tested resistant strain (FIGS. 3C and 3D). These results demonstrated that resistance-targeting adjuvants would prove insufficient against tolerant bacteria, highlighting the clinical significance of identifying and combating bacterial tolerance mechanisms.

β-lactams, especially penicillins, have been previously described to exhibit only limited efficacy against Mycobacteria, which in addition to expression of genomic β-lactamases also possess altered peptidoglycan structure, with increased levels of 3-3 crosslinks formed by L,D-transpeptidases (23). Efficacy of β-lactams, which primarily target D,D-transpeptidases, can be enhanced by simultaneously targeting L,D-transpeptidases (24, 25). In view of the instant findings, it was posited that D-methionine might also potentiate ampicillin in Mycobacterium smegmatis, further noting that L,D-transpeptidases have been described as incorporating D-alanine analogues into peptidoglycan in Mycobacterium smegmatis (26) and as incorporating NCDAAs into peptidoglycan in other species (10). As observed with the resistant clinical isolates of E. coli and K. pneumoniae tested above, the combination of a carbon source, D-methionine, and sulbactam sensitized M. smegmatis to ampicillin (FIG. 3F). Here, glucose was used as the tested carbon source, rather than pyruvate, because glucose was previously used to sensitize M. smegmatis to quinolone antibiotics (11), though it was herein identified that glycerol was also effective at provoking such sensitization (FIG. 3G). Sensitization of M. smegmatis to amoxicillin-clavulanic acid was also observed (FIGS. 3H and 3I). The current results have therefore identified the efficacy of D-amino acid supplementation (in combination with a carbon source and a β-lactamase inhibitor) in sensitizing Mycobacterium to β-lactam antibiotics. Noting the globally increasing levels of multidrug resistant Mycobacterium tuberculosis reported in recent years, it is expressly contemplated that treatment as described herein can expand the functional repertoire of β-lactam antibiotics.

In conclusion, the above examples have demonstrated that metabolic stimulation was able to restore β-lactam sensitivity in tolerant, stationary phase bacteria and that such metabolic stimulation-induced effects could be potentiated through the drug-specific effects of non-canonical D-amino acids. Additionally growth-independent effects of D-alanine and D-serine supplementation were observed, and it was also discovered that many D-amino acids potentiated sensitivity of reawakened bacteria. It was also shown that in the case of nutrient limitation and β-lactamase expression, tolerance and resistance were orthogonal phenotypes, and that both of which needed to be addressed to restore sensitivity to β-lactams. Finally, the instant approach was identified as generalizable to multiple conditions and multiple bacterial species.

### REFERENCES

1. Baym, M., Stone, L. K. & Kishony, R. Multidrug evolutionary strategies to reverse antibiotic resistance. Science 351, aad3292 (2016).
2. Wright, G. D. Antibiotic Adjuvants: Rescuing Antibiotics from Resistance: (Trends in Microbiology 24, 862-871; Oct. 17, 2016). Trends Microbiol. 24, 928 (2016).
3. Drawz, S. M. & Bonomo, R. A. Three decades of beta-lactamase inhibitors. Clin. Microbiol. Rev. 23, 160-201 (2010).
4. Imamovic, L. et al. Drug-Driven Phenotypic Convergence Supports Rational Treatment Strategies of Chronic Infections. Cell 172, 121-134.e14 (2018).
5. Brauner, A., Fridman, O., Gefen, O. & Balaban, N. Q. Distinguishing between resistance, tolerance and persistence to antibiotic treatment. Nat. Rev. Microbiol. 14, 320-330 (2016).
6. Meylan, S., Andrews, I. W. & Collins, J. J. Targeting Antibiotic Tolerance, Pathogen by Pathogen. Cell 172, 1228-1238 (2018).
7. Levin-Reisman, I. et al. Antibiotic tolerance facilitates the evolution of resistance. Science 355, 826-830 (2017).
8. Yang, J. H., Bening, S. C. & Collins, J. J. Antibiotic efficacy-context matters. Curr. Opin. Microbiol. 39, 73-80 (2017).
9. Radzikowski, J. L. et al. Bacterial persistence is an active GS stress response to metabolic flux limitation. Mol. Syst. Biol. 12, 882 (2016).
10. Cava, F., de Pedro, M. A., Lam, H., Davis, B. M. & Waldor, M. K. Distinct pathways for modification of the bacterial cell wall by non-canonical D-amino acids. EMBO J. 30, 3442-3453 (2011).
11. Gutierrez, A. et al. Understanding and Sensitizing Density-Dependent Persistence to Quinolone Antibiotics. Mol. Cell 68, 1147-1154.e3 (2017).
12. Cho, H., Uehara, T. & Bernhardt, T. G. Beta-lactam antibiotics induce a lethal malfunctioning of the bacterial cell wall synthesis machinery. Cell 159, 1300-1311 (2014).
13. Blasco, B., Pisabarro, A. G. & de Pedro, M. A. Peptidoglycan biosynthesis in stationary-phase cells of Escherichia coli. J. Bacteriol. 170, 5224-5228 (1988).
14. Caparrós, M., Pisabarro, A. G. & de Pedro, M. A. Effect of D-amino acids on structure and synthesis of peptidoglycan in Escherichia coli. J. Bacteriol. 174, 5549-5559 (1992).
15. Tsuruoka, T. et al. Penicillin-insensitive incorporation of D-amino acids into cell wall peptidoglycan influences the amount of bound lipoprotein in Escherichia coli. J. Bacteriol. 160, 889-894 (1984).
16. Tsuruoka, T. et al. Second lytic target of beta-lactam compounds that have a terminal d-amino acid residue. Eur. J. Biochem. 151, 209-216 (1985).
17. Prax, M., Mechler, L., Weidenmaier, C. & Bertram, R. Glucose Augments Killing Efficiency of Daptomycin Challenged Staphylococcus aureus Persisters. PLoS One 11, e0150907 (2016).
18. Meylan, S. et al. Carbon Sources Tune Antibiotic Susceptibility in Pseudomonas aeruginosa via Tricarboxylic Acid Cycle Control. Cell Chem Biol 24, 195-206 (2017).
19. Allison, K. R., Brynildsen, M. P. & Collins, J. J. Metabolite-enabled eradication of bacterial persisters by aminoglycosides. Nature 473, 216-220 (2011).
20. Peng, B. et al. Exogenous Alanine and/or Glucose plus Kanamycin Kills Antibiotic-Resistant Bacteria. Cell Metab. 21, 249-262 (2015).
21. Bucher, T., Oppenheimer-Shaanan, Y., Savidor, A., Bloom-Ackermann, Z. & Kolodkin-Gal, I. Disturbance of the bacterial cell wall specifically interferes with biofilm formation. Environ. Microbiol. Rep. 7, 990-1004 (2015).
22. Aliashkevich, A., Alvarez, L. & Cava, F. New Insights Into the Mechanisms and Biological Roles of D-Amino Acids in Complex Eco-Systems. Front. Microbiol. 9, 683 (2018).
23. Story-Roller, E. & Lamichhane, G. Have we realized the full potential of β-lactams for treating drug-resistant TB? IUBMB Life 70, 881-888 (2018).
24. Hugonnet, J.-E., Tremblay, L. W., Boshoff, H. I., Barry, C. E., 3rd & Blanchard, J. S. Meropenem-clavulanate is effective against extensively drug-resistant Mycobacterium tuberculosis. Science 323, 1215-1218 (2009).
25. Gupta, R. et al. The Mycobacterium tuberculosis protein LdtMt2 is a nonclassical transpeptidase required for virulence and resistance to amoxicillin. Nat. Med. 16, 466-469 (2010).
26. Baranowski, C. et al. Maturing Mycobacterial Peptidoglycan Requires Non-canonical Crosslinks to Maintain Shape. (2018). doi: 10.1101/291823.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A pharmaceutical composition comprising:
(a) a metabolic stimulant;
(b) a D-amino acid;
(c) an antibiotic or a pharmaceutically acceptable salt thereof,
and a pharmaceutically acceptable carrier.

2. The pharmaceutical composition of claim 1, wherein the metabolic stimulant is a carbon source, optionally wherein the metabolic stimulant is selected from the group consisting of citrate, propionic acid, succinate, pyruvate, fumarate, acetate, mannitol, glycerol, arabinose, fructose, xylose, glucose, ribose, gluconate, a L-amino acid and a D-amino acid, optionally wherein the L-amino acid is L-serine or L-alanine or wherein the D-amino acid is D-serine or D-alanine.

3. The pharmaceutical composition of claim 1, wherein the D-amino acid is selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-norleucine, D-glutamine, D-arginine, D-serine, D-threonine, D-valine, D-tryptophan, D-asparagine, D-phenylglycine, D-tyrosine, D-alpha-aminobutyric acid, and D-alpha-aminopimelic acid.

4. The pharmaceutical composition of claim 1, wherein the antibiotic is selected from the group consisting of:
(a) a β-lactam antibiotic, an aminoglycoside antibiotic and/or a quinolone antibiotic, optionally wherein the β-lactam antibiotic is selected from the group consisting of:
a penicillin derivative (e.g., Benzathine penicillin (benzathine & benzylpenicillin), Benzylpenicillin (penicillin G), Phenoxymethylpenicillin (penicillin V), Procaine penicillin (procaine & benzylpenicillin), Pheneticillin, Cloxacillin, Dicloxacillin, Flucloxacillin, Methicillin, Nafcillin, Oxacillin, Temocillin, Amoxicillin, Ampicillin, Mecillinam, Carbenicillin, Ticarcillin, Azlocillin, Mezlocillin, and Piperacillin),
a cephalosporin (e.g., Cefazolin, Cephalexin, Cephalosporin C, Cephalothin, Cefaclor, Cefamandole, Cefuroxime, Cefotetan, Cefoxitin, Cefixime, Cefotaxime, Cefpodoxime, Ceftazidime, Ceftriaxone, Cefepime, Cefpirome, Cefsulodin and Ceftaroline),
a monobactam (e.g., Aztreonam, Tigemonam, Nocardicin A, and Tabtoxinine β-lactam), and
a carbapenem or penem (e.g., Biapenem, Doripenem, Ertapenem, Faropenem, Imipenem, Meropenem, Panipenem, Razupenem, Tebipenem, and Thienamycin), and/or
(b) a non-β-lactam cell wall-active antibiotic, optionally wherein the non-β-lactam cell wall-active antibiotic is selected from the group consisting of a NAM synthesis inhibitor (e.g., Fosfomycin), a DADAL/AR inhibitor (e.g., Cycloserine), a bactoprenol inhibitor (e.g., Bacitracin), a PG chain elongation inhibitor (e.g., Vancomycin (Oritavancin, Telavancin), Teicoplanin (Dalbavancin), Ramoplanin), a polymyxin/detergent (e.g., Colistin, Polymyxin B), a depolarizing agent (e.g., Daptomycin), a NAM-NAG hydrolysis agent (e.g., lysozyme), a Tyrothricin (e.g., Gramicidin, Tyrocidine), Isoniazid, and/or Teixobactin.

5. The pharmaceutical composition of claim 1, further comprising a β-lactamase inhibitor.

6. The pharmaceutical composition of claim 5, wherein the β-lactamase inhibitor is selected from the group consisting of sulbactam, tebipenem, a Boron based transition state inhibitor (e.g., Ec19), clavulanic acid, tazobactam, avibactam and relebactam.

7. The pharmaceutical composition of claim 1, wherein the antibiotic is present in an amount between 0.1 g and 2.0 g.

8. The pharmaceutical composition of claim 1, wherein the D-amino acid is provided in an amount sufficient to potentiate the antibiotic to kill at least 80% of a target population of bacteria that possess antibiotic tolerance.

9. A composition selected from the group consisting of:
A pharmaceutical composition comprising: (a) a β-lactamase inhibitor; (b) a metabolic stimulant and/or a D-amino acid; and a pharmaceutically acceptable carrier; and
A kit comprising a metabolic stimulant, a D-amino acid, a β-lactam antibiotic, and instructions for its use.

10. A method selected from the group consisting of:
A method for sensitizing a bacteria to an antibiotic comprising contacting the bacteria with a metabolic stimulant and a D-amino acid, thereby sensitizing the bacteria to the antibiotic;
A method for sensitizing a bacteria that expresses β-lactamase to an antibiotic comprising contacting the bacteria with a β-lactamase inhibitor and a metabolic stimulant and/or a D-amino acid, thereby sensitizing the bacteria to the antibiotic;
A method for treating or preventing a bacterial infection in a subject comprising administering a pharmaceutical composition comprising (a) a metabolic stimulant (b) a D-amino acid; (c) an antibiotic or a pharmaceutically acceptable salt thereof, and a pharmaceutically acceptable carrier to a subject having or at risk of developing a bacterial infection, thereby treating or preventing the bacterial infection in the subject; and
A method for treating or preventing a bacterial infection in a subject, wherein bacteria of the bacterial infection express β-lactamase and exhibit antibiotic tolerance, the method comprising administering a pharmaceutical composition comprising (a) a β-lactamase inhibitor; (b) a metabolic stimulant and/or a D-amino acid; and (c) a pharmaceutically acceptable carrier, in an amount sufficient to treat or prevent the bacterial infection in the subject, thereby treating or preventing the bacterial infection in the subject.

11. The method of claim 10, wherein the bacteria exhibits tolerance to the antibiotic.

12. The method of claim 10, wherein the bacteria is selected from the group consisting of Escherichia coli, Klebsiella and Mycobacteria, optionally wherein the Klebsiella is a Klebsiella pneumoniae or wherein the Mycobacteria is a Mycobacterium smegmatis or a Mycobacterium tuberculosis.

13. The method of claim 10, wherein the D-amino acid is selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-norleucine, D-glutamine, D-arginine, D-serine, D-threonine, D-valine, D-tryptophan, D-asparagine, D-phenylglycine, D-tyrosine, D-alpha-aminobutyric acid, and D-alpha-aminopimelic acid.

14. The method of claim 10, wherein the metabolic stimulant is a carbon source, optionally wherein the metabolic stimulant is selected from the group consisting of citrate, propionic acid, succinate, pyruvate, fumarate, acetate, mannitol, glycerol, arabinose, fructose, xylose, glucose, ribose, gluconate, a L-amino acid and a D-amino acid, optionally wherein the L-amino acid is L-serine or L-alanine or wherein the D-amino acid is D-serine or D-alanine.

15. The method of claim 10, wherein the antibiotic is selected from the group consisting of:
(a) a β-lactam antibiotic, an aminoglycoside antibiotic and/or a quinolone antibiotic, optionally wherein the β-lactam antibiotic is selected from the group consisting of:
a penicillin derivative (e.g., Benzathine penicillin (benzathine & benzylpenicillin), Benzylpenicillin (penicillin G), Phenoxymethylpenicillin (penicillin V), Procaine penicillin (procaine & benzylpenicillin), Pheneticillin, Cloxacillin, Dicloxacillin, Flucloxacillin, Methicillin, Nafcillin, Oxacillin, Temocillin, Amoxicillin, Ampicillin, Mecillinam, Carbenicillin, Ticarcillin, Azlocillin, Mezlocillin, and Piperacillin),
a cephalosporin (e.g., Cefazolin, Cephalexin, Cephalosporin C, Cephalothin, Cefaclor, Cefamandole, Cefuroxime, Cefotetan, Cefoxitin, Cefixime, Cefotaxime, Cefpodoxime, Ceftazidime, Ceftriaxone, Cefepime, Cefpirome, Cefsulodin and Ceftaroline),
a monobactam (e.g., Aztreonam, Tigemonam, Nocardicin A, and Tabtoxinine β-lactam), and
a carbapenem or penem (e.g., Biapenem, Doripenem, Ertapenem, Faropenem, Imipenem, Meropenem, Panipenem, Razupenem, Tebipenem, and Thienamycin), and/or
(b) a non-β-lactam cell wall-active antibiotic, optionally wherein the non-β-lactam cell wall-active antibiotic is selected from the group consisting of a NAM synthesis inhibitor (e.g., Fosfomycin), a DADAL/AR inhibitor (e.g., Cycloserine), a bactoprenol inhibitor (e.g., Bacitracin), a PG chain elongation inhibitor (e.g., Vancomycin (Oritavancin, Telavancin), Teicoplanin (Dalbavancin), Ramoplanin), a polymyxin/detergent (e.g., Colistin, Polymyxin B), a depolarizing agent (e.g., Daptomycin), a NAM-NAG hydrolysis agent (e.g., lysozyme), a Tyrothricin (e.g., Gramicidin, Tyrocidine), Isoniazid, and/or Teixobactin.

16. The method of claim 10, further comprising contacting the bacteria with a β-lactamase inhibitor.

17. The method of claim 10, wherein the β-lactamase inhibitor is selected from the group consisting of sulbactam, tebipenem, a Boron based transition state inhibitor (e.g., Ec19), clavulanic acid, tazobactam, avibactam and relebactam.

18. (canceled)

19. The method of claim 10, wherein the bacteria exhibits tolerance to the antibiotic.

20. The method of claim 10, wherein:
the bacteria is selected from the group consisting of Escherichia coli, Klebsiella and Mycobacteria, optionally wherein the Klebsiella is a Klebsiella pneumoniae or wherein the Mycobacteria is a Mycobacterium smegmatis or a Mycobacterium tuberculosis;
the D-amino acid is selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-norleucine, D-glutamine, D-arginine, D-serine, D-threonine, D-valine, D-tryptophan, D-asparagine, D-phenylglycine, D-tyrosine, D-alpha-aminobutyric acid, and D-alpha-aminopimelic acid;
the metabolic stimulant is a carbon source, optionally wherein the metabolic stimulant is selected from the group consisting of citrate, propionic acid, succinate, pyruvate, fumarate, acetate, mannitol, glycerol, arabinose, fructose, xylose, glucose, ribose, gluconate, a L-amino acid and a D-amino acid, optionally wherein the L-amino acid is L-serine or L-alanine or wherein the D-amino acid is D-serine or D-alanine;
the antibiotic is selected from the group consisting of (a) a β-lactam antibiotic, an aminoglycoside antibiotic and/or a quinolone antibiotic, optionally wherein the β-lactam antibiotic is selected from the group consisting of a penicillin derivative (e.g., Benzathine penicillin (benzathine & benzylpenicillin), Benzylpenicillin (penicillin G), Phenoxymethylpenicillin (penicillin V), Procaine penicillin (procaine & benzylpenicillin), Pheneticillin, Cloxacillin, Dicloxacillin, Flucloxacillin, Methicillin, Nafcillin, Oxacillin, Temocillin, Amoxicillin, Ampicillin, Mecillinam, Carbenicillin, Ticarcillin, Azlocillin, Mezlocillin, and Piperacillin), a cephalosporin (e.g., Cefazolin, Cephalexin, Cephalosporin C, Cephalothin, Cefaclor, Cefamandole, Cefuroxime, Cefotetan, Cefoxitin, Cefixime, Cefotaxime, Cefpodoxime, Ceftazidime, Ceftriaxone, Cefepime, Cefpirome, Cefsulodin and Ceftaroline), a monobactam (e.g., Aztreonam, Tigemonam, Nocardicin A, and Tabtoxinine β-lactam), and a carbapenem or penem (e.g., Biapenem, Doripenem, Ertapenem, Faropenem, Imipenem, Meropenem, Panipenem, Razupenem, Tebipenem, and Thienamycin), and/or (b) a non-β-lactam cell wall-active antibiotic, optionally wherein the non-β-lactam cell wall-active antibiotic is selected from the group consisting of a NAM synthesis inhibitor (e.g., Fosfomycin), a DADAL/AR inhibitor (e.g., Cycloserine), a bactoprenol inhibitor (e.g., Bacitracin), a PG chain elongation inhibitor (e.g., Vancomycin (Oritavancin, Telavancin), Teicoplanin (Dalbavancin), Ramoplanin), a polymyxin/detergent (e.g., Colistin, Polymyxin B), a depolarizing agent (e.g., Daptomycin), a NAM-NAG hydrolysis agent (e.g., lysozyme), a Tyrothricin (e.g., Gramicidin, Tyrocidine), Isoniazid, and/or Teixobactin;
the method further comprises contacting the bacteria with a β-lactamase inhibitor;
the β-lactamase inhibitor is selected from the group consisting of sulbactam, tebipenem, a Boron based transition state inhibitor (e.g., Ec19), clavulanic acid, tazobactam, avibactam and relebactam;
the subject is human; and/or
the bacterial infection is an antibiotic tolerant bacterial infection.

21-31. (canceled)

32. The composition of claim 9, wherein the kit further comprises a β-lactamase inhibitor, optionally wherein the β-lactamase inhibitor is selected from the group consisting of sulbactam, tebipenem, a Boron based transition state inhibitor (e.g., Ec19), clavulanic acid, tazobactam, avibactam and relebactam.
